(19) United States
(12) Patent Application Publication
(10) Pub. No.: US 2025/0235562 A1
(43) Pub. Date: Jul. 24, 2025

(54) USE OF SECOND NEAR-INFRARED ORGANIC FLUORESCENT COMPOUND IN PREPARATION OF BIOIMAGING CONTRAST AGENT AND IN VASCULAR FLUORESCENCE IMAGING

(71) Applicant: INNER MONGOLIA UNIVERSITY, Huhhot (CN)

(72) Inventors: Jianguo WANG, Huhhot (CN); Guoyu JIANG, Huhhot (CN); Chunbin LI, Huhhot (CN)

(21) Appl. No.: 18/781,717

(22) Filed: Jul. 23, 2024

(30) Foreign Application Priority Data
Jan. 23, 2024 (CN) 202410088799.8
Feb. 2, 2024 (WO) PCT/CN2024/075483

(51) Int. Cl. A61K 49/00; C07D 513/22

(52) U.S. Cl. CPC A61K 49/0021; A61K 49/0054; C07D 513/22; A61K 2123/00

(57) ABSTRACT

Disclosed is use of a second near-infrared (NIR-II) organic fluorescent compound in the preparation of a bioimaging contrast agent and in vascular fluorescence imaging. The NIR-II organic fluorescent compound having the structure represented by formula I could be used to construct a high-performance bioimaging contrast agent, and shows broad prospects in the field of in vivo imaging and fluorescent surgical navigation.

### CROSS REFERENCE TO RELATED APPLICATION

The present application is a continuation-in-part application of International Patent Application No. PCT/CN2024/075483, filed on Feb. 2, 2024, which claims priority to Chinese Patent Application No. 202410088799.8, entitled “USE OF SECOND NEAR-INFRARED FLUORESCENT ORGANIC COMPOUND IN PREPARATION OF BIOIMAGING CONTRAST AGENT AND IN VASCULAR FLUORESCENCE IMAGING”, and filed with the China National Intellectual Property Administration on Jan. 23, 2024. The disclosure of the two applications is incorporated by references herein in their entireties as part of the present application.

### TECHNICAL FIELD

The present disclosure belongs to the technical field of biochemical materials, and specifically relates to use of a second near-infrared organic fluorescent compound in the preparation of a bioimaging contrast agent and in vascular fluorescence imaging.

### BACKGROUND

In vivo imaging technology refers to a technology for qualitative and quantitative study on biological processes in vivo at the tissue, cellular, and molecular levels without damaging animals using imaging methods. This technology allows for non-invasive and intuitive observation of various biological processes in living animals, which is of great significance for fields such as life sciences and medicine. Among them, in vivo fluorescence imaging technology has received widespread attention due to its high sensitivity, high spatiotemporal resolution, and real-time monitoring ability. As the core of in vivo fluorescence imaging technology, high-performance fluorescence imaging agents have always been a research focus for researchers. Second near-infrared (NIR-II) organic fluorescent molecules have become an ideal choice for high-performance fluorescence imaging agents due to their greater tissue penetration depth and low spontaneous fluorescence interference. However, when using NIR-II organic nanoimaging agents for in vivo fluorescence imaging, expensive lasers with specific wavelength(s) are needed to be used as excitation light sources, because of the long absorption wavelength, low quantum yield, and limited molar extinction coefficient of NIR-II organic fluorescence molecules. Furthermore, the use of single-wavelength lasers also brings about problems such as limited photon absorption and laser-induced biological damage. In addition, single-wavelength lasers could not provide uniform irradiation, and the energy provided thereby would decay from the center of the spot to the surrounding, which would also adversely affect the imaging quality of in vivo imaging.

In contrast, white light, including ordinary lamps, surgical shadowless lamps, and laparoscopic light sources, as a safe and visible excitation light source, has the characteristics of a wide continuous spectrum, low cost, and easy availability, and is an ideal choice for excitation light sources in in vivo fluorescence imaging. However, use of white light as an excitation light source in NIR-II fluorescence imaging has not been reported yet, which requires NIR-II organic nanoimaging agents to have high quantum yields and molar extinction coefficients. Therefore, the development of high-performance NIR-II organic nanoimaging agents excited by white light is urgently needed, but faces great challenges.

### SUMMARY

An object of the present disclosure is to provide use of a second near-infrared organic fluorescent compound in the preparation of a bioimaging contrast agent and in vascular fluorescence imaging. The second near-infrared organic fluorescent compound according to the present disclosure has good biocompatibility and biological stability, and a high molar extinction coefficient and a high quantum yield, which could achieve high-resolution imaging of blood vessels under white light excitation. The second near-infrared organic fluorescent compound according to the present disclosure could be used to construct a high-performance bioimaging contrast agent, and shows broad prospects in the field of non-therapeutic in vivo imaging and fluorescent surgical navigation.

In order to achieve the above object, the present disclosure provides the following technical solutions:

Provided is use of a second near-infrared (NIR-II) organic fluorescent compound in the preparation of a bioimaging contrast agent, wherein the NIR-II organic fluorescent compound has a structure represented by formula I,

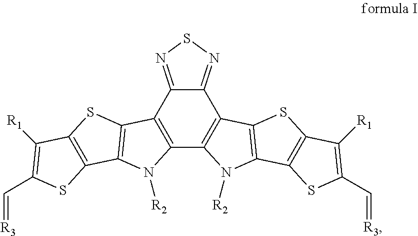

wherein in formula I,
R₁ and R₂ each represent branched or linear alkyl; and
R₃ represents

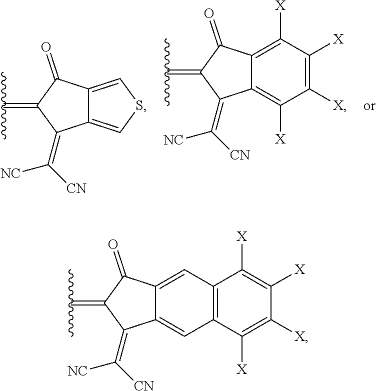

X being at least one selected from the group consisting of H, F, and Cl.

In some embodiments, R₁ and R₂ each represent C₁-C₁₁ branched or linear alkyl.

In some embodiments, the NIR-II organic fluorescent compound has a structure represented by formula I-a, formula I-b, or formula I-c,

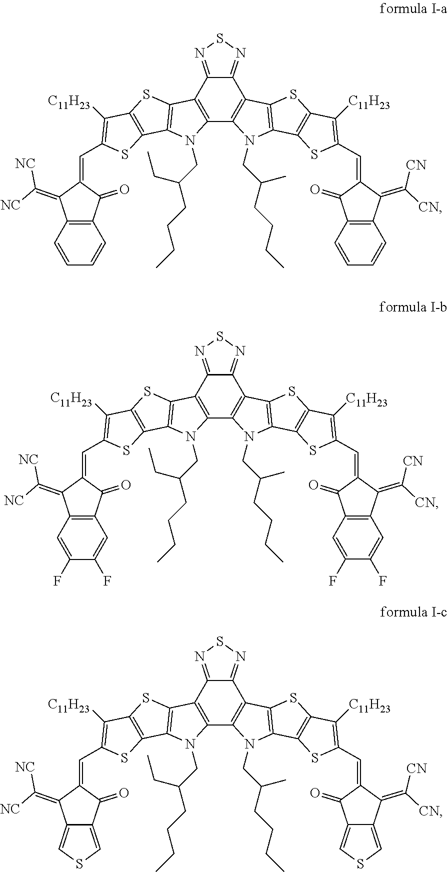

wherein in formula I-a, formula I-b, or formula I-c, C₁₁-23 is linear alkyl.

Further provided is use of an NIR-II organic fluorescent compound in non-diagnostic and non-therapeutic vascular fluorescence imaging, wherein the NIR-II organic fluorescent compound has a structure represented by formula I,

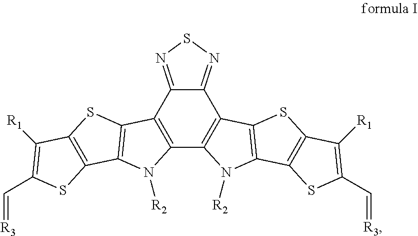

wherein in formula I,
R₁ and R₂ each represent branched or linear alkyl; and
R₃ represents

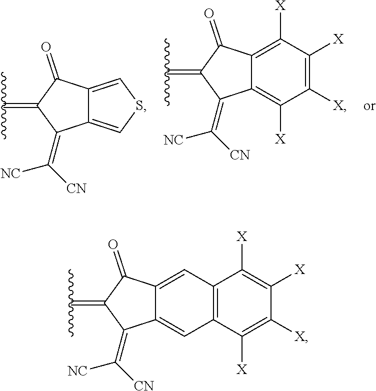

X being at least one selected from the group consisting of H, F, and Cl.

Further provided is use of a nanoimaging agent in the preparation of a bioimaging contrast agent, wherein the nanoimaging agent includes an NIR-II organic fluorescent compound having a structure represented by formula I, and an organic encapsulation matrix covering a surface of the NIR-II organic fluorescent compound having the structure represented by formula I,

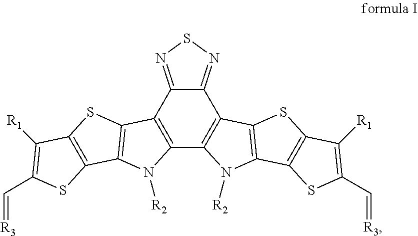

wherein in formula I,
R₁ and R₂ each represent branched or linear alkyl; and
R₃ represents

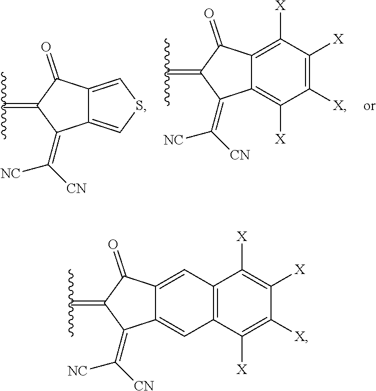

X in R₃ being at least one selected from the group consisting of H, F, and Cl.

In some embodiments, the organic encapsulation matrix includes at least one selected from the group consisting of methoxypolyethylene glycol amine, 1,2-distearoyl-sn-glycero-3-phosphoethanolamine-poly(ethylene glycol), 1,2-distearoyl-sn-glycero-3-phosphoethanolamine-N-[maleimide(polyethylene glycol)], 1,2-distearoyl-sn-glycero-3-phosphoethanolamine-N-[folate(polyethylene glycol)], 1,2-distearoyl-sn-glycero-3-phosphoethanolamine-N-[thiol(polyethylene glycol)], 1,2-distearoyl-sn-glycero-3-phosphoethanolamine-N-[carboxy(polyethylene glycol)], 1,2-distearoyl-sn-glycero-3-phosphoethanolamine-N-[azido(polyethylene glycol)], 1,2-distearoyl-sn-glycero-3-ethanolamine-N-[biotin(polyethylene glycol)], 1-palmitoyl-2-oleoyl-sn-glycero-3-phosphoethanolamine, 1-stearoyl-2-oleoyl lecithin, 1,2-dipalmitoyl phosphoethanolamine-polyethylene glycol, polystyrene-grafted-poly(ethylene glycol), methoxy PEG polylactic acid-hydroxyacetic acid copolymer, and Pluronic® F-127.

In some embodiments, the nanoimaging agent is prepared by a method including the steps of
mixing the organic encapsulation matrix, the NIR-II organic fluorescent compound having the structure represented by formula I, and an organic solvent to obtain a mixed solution; and mixing the mixed solution with water, and subjecting a resulting mixture to ultrasonic assembly, to obtain an assembly liquid;
introducing the assembly liquid into a dialysis bag and performing dialysis, to obtain a purified assembly material; and
concentrating the purified assembly material, to obtain a solution of the nanoimaging agent.

In some embodiments, a mass ratio of the organic encapsulation matrix to the NIR-II organic fluorescent compound having the structure represented by formula I is in a range of 3:1 to 8:1; and
the dialysis bag has a molecular weight cut-off of 3500, and the dialysis is performed for 48-72 h.

Provided is use of a nanoimaging agent in non-diagnostic and non-therapeutic vascular fluorescence imaging, wherein the nanoimaging agent includes an NIR-II organic fluorescent compound having a structure represented by formula I, and an organic encapsulation matrix covering a surface of the NIR-II organic fluorescent compound having the structure represented by formula I,

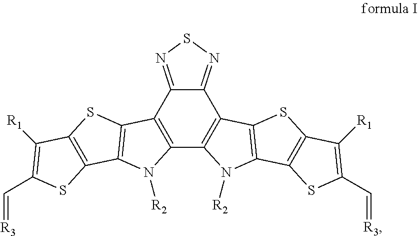

wherein in formula I,
R₁ and R₂ each represent branched or linear alkyl; and
R₃ represents

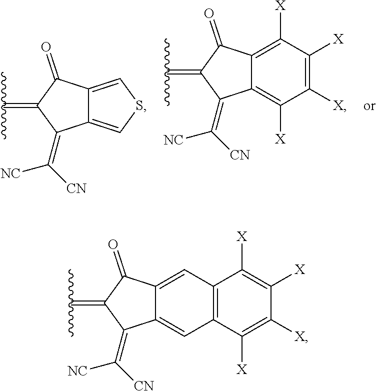

X in R₃ being at least one selected from the group consisting of H, F, and Cl.

In some embodiments, the vascular fluorescence imaging is performed under excitation of white light, the white light having a wavelength of 400-800 nm.

The present disclosure provides use of an NIR-II organic fluorescent compound in the preparation of a bioimaging contrast agent, wherein the NIR-II organic fluorescent compound has a structure represented by formula I. The NIR-II organic fluorescent compound having the structure represented by formula I according to the present disclosure has a high molar extinction coefficient and a high quantum yield, and could achieve high-resolution imaging of blood vessels under white light excitation. Furthermore, the NIR-II organic fluorescent compound having the structure represented by formula I could visually monitor the liver ischemia-reperfusion process and the kidney transplantation process. In addition, the NIR-II organic fluorescent compound having the structure represented by formula I according to the present disclosure could effectively avoid problems such as limited photon absorption, laser-induced biological damage, and uneven irradiation caused by single-wavelength excitation light source by using white light as the excitation light source in the imaging process. Therefore, the NIR-II organic fluorescent compound having the structure represented by formula I according to the present disclosure could be used to construct high-performance contrast agents for bioimaging, with broad prospects in the field of non-diagnostic and non-therapeutic in vivo imaging and fluorescent surgical navigation.

The present disclosure provides use of a nanoimaging agent in the preparation of a bioimaging contrast agent, wherein the nanoimaging agent includes an NIR-II organic fluorescent compound having the structure represented by formula I, and an organic encapsulation matrix covering a surface of the NIR-II organic fluorescent compound having the structure represented by formula I. In the present disclosure, the nanoimaging agent is obtained from the NIR-II organic fluorescent compound and the organic encapsulation matrix, and has good biocompatibility and biological stability. The nanoimaging agent according to the present disclosure has a high molar extinction coefficient and a high quantum yield, and could achieve high-resolution imaging of blood vessels under white light excitation. Furthermore, the nanoimaging agent could visually monitor the liver ischemia-reperfusion process and the kidney transplantation process. In addition, the use of white light as the excitation light source in the imaging process could effectively avoid problems such as limited photon absorption, laser-induced biological damage, and uneven irradiation caused by single-wavelength excitation light sources. Therefore, the nanoimaging agent described in the present disclosure could be used to construct high-performance contrast agents for biological imaging, with broad prospects in the field of non-therapeutic in vivo imaging and fluorescent surgical navigation.

The results of the examples indicate that the nanoimaging agent described in the present disclosure has uniform size, good stability, low toxic side effects, NIR-II fluorescence emission, and a high quantum yield. Thus, the nanoimaging agent could effectively increase the penetration depth of biological tissues, reduce interference of spontaneous fluorescence of biological tissues, and improve a signal-to-noise ratio and an imaging resolution. When applied to abdominal blood vessels in mice for imaging, this nanoimaging agent could achieve rapid and high-resolution imaging of abdominal blood vessels in mice. When using the nanoimaging agent described in the present disclosure to monitor the liver ischemia-reperfusion process in mice, the experimental results show that the nanoimaging agent could achieve rapid, real-time, and high-resolution imaging of this process. In addition, the nanoimaging agent described in the present disclosure could also be applied to monitor the kidney transplantation process in a New Zealand white rabbit, and achieve real-time high-resolution imaging of changes in renal blood vessels during the kidney transplantation. It is notable that due to the excellent luminescent properties and imaging ability of the nanoimaging agent described in the present disclosure, all excitation light sources used in in vivo fluorescence imaging applications are white light sources. White light sources are not only cheaper and more economical, but also could effectively avoid problems caused by single-wavelength excitation light sources, such as limited photon absorption, laser-induced biological damage, and uneven irradiation. Therefore, the nanoimaging agent described in the present disclosure could be used to construct high-performance contrast agents for bioimaging, with broad prospects in the field of non-therapeutic and non-diagnostic purpose in in vivo imaging.

### BRIEF DESCRIPTION OF THE DRAWINGS

FIG. 1A to FIG. 1C show particle sizes of the nanoimaging agents HY6-NPs, FY6-NPs, and Y6CT-NPs, respectively.

FIG. 1D shows a transmission electron microscope (TEM) image of the nanoimaging agent Y6CT-NPs.

FIG. 2A shows absorption spectra of the nanoimaging agents HY6-NPs, FY6-NPs, and Y6CT-NPs in aqueous solution.

FIG. 2B shows emission spectra of the nanoimaging agents HY6-NPs, FY6-NPs, and Y6CT-NPs in aqueous solution.

FIG. 3 shows a comparison of the photostability between the nanoimaging agent Y6CT-NPs and the commercial imaging agent indocyanine green (ICG).

FIG. 4A to FIG. 4C show relative quantum yield test results of the nanoimaging agents FY6-NPs, HY6-NPs, and Y6CT-NPs, respectively, with commercial dye IR26 as a reference.

FIG. 5 shows the dark toxicity and phototoxicity of Y6CT-NPs at different concentrations on human normal liver cells (LO2 cells).

FIG. 6 shows the dark toxicity and phototoxicity of Y6CT-NPs at different concentrations on mouse embryonic fibroblasts (NIH 3T3 cells).

FIG. 7A to FIG. 7D show the NIR-II fluorescence imaging of normal mouse abdominal blood vessels using the nanoimaging agent Y6CT-NPs under different filter conditions.

FIG. 7E, FIG. 7F, and FIG. 7G show resolution analysis graphs in the NIR-II fluorescence imaging of normal mouse abdominal blood vessels using the nanoimaging agent Y6CT-NPs under different filter conditions.

FIG. 8A to FIG. 8D show a real-time NIR-II fluorescence image of a mouse liver during ischemia-reperfusion process using nanoimaging agent Y6CT-NPs.

FIG. 9 shows fluorescence intensity analysis of different regions in mouse liver during ischemia-reperfusion process monitored using Y6CT-NPs.

FIG. 10A to FIG. 10F show a real-time NIR-II fluorescence image of the donor kidney region in a New Zealand white rabbit using the nanoimaging agent Y6CT-NPs.

FIG. 11A to FIG. 11D show a real-time monitoring image of anastomosis of renal blood vessel during the kidney transplantation in a New Zealand white rabbit using the nanoimaging agent Y6CT-NPs.

FIG. 12A to FIG. 12F show a real-time monitoring image of blood supply to ureter in the transplanted kidney during the kidney transplantation in New Zealand white rabbits, using the nanoimaging agent Y6CT-NPs.

### DETAILED DESCRIPTION OF THE EMBODIMENTS

The present disclosure provides use of an NIR-II organic fluorescent compound in the preparation of a bioimaging contrast agent, wherein the NIR-II organic fluorescent compound has a structure represented by formula I,

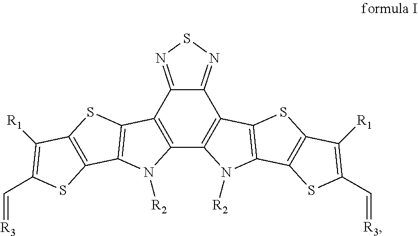

wherein in formula I,
R₁ and R₂ each represent branched or linear alkyl; and
R₃ represents

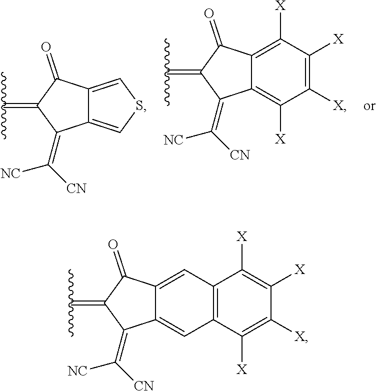

X being at least one selected from the group consisting of H, F, and Cl.

In the present disclosure, unless otherwise specified, all preparation raw materials/components are commercially available products that are well-known to those skilled in the art.

In some embodiments of the present disclosure, R₁ and R₂ each represent C₁-C₁₁ branched or linear alkyl, preferably C₅-C₁₁ branched or linear alkyl, and more preferably C₁₁ branched or linear alkyl.

In some embodiments of the present disclosure, R₃ represents

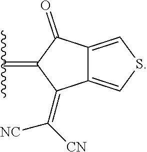 [SMILES: *=C1C(=O)c2cscc2C1=C(C#N)C#N]

In some embodiments of the present disclosure, R₃ represents

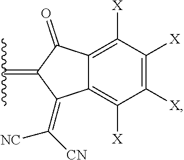

X being H, or X being H and F.

In some embodiments of the present disclosure, the NIR-II organic fluorescent compound has a structure represented by formula I-a, formula I-b, or formula I-c,

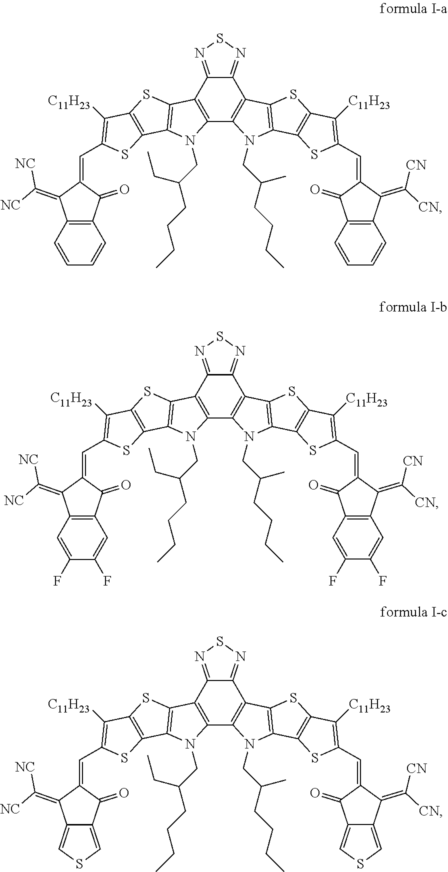

wherein in formula I-a, formula I-b, or formula I-c, C₁₁-23 is linear alkyl.

In specific embodiments of the present disclosure, the NIR-II organic fluorescent compound having a structure represented by formula I-a and the NIR-II organic fluorescent compound having a structure represented by formula I-b each are prepared according to “Fluorination Enhances NIR-II Emission and Photothermal Conversion Efficiency of Phototheranostic Agents for Imaging-Guided Cancer Therapy” (Chunbin Li, Guoyu Jiang, Jia Yu, Weiwei Ji, Lingxiu Liu, Pengfei Zhang, Jian Du, Chuanlang Zhan, Jianguo Wang, and Ben Zhong Tang., Advanced Materlals, 2023, 35, 2208229-2208239, incorporated by reference).

In specific embodiments of the present disclosure, the NIR-II organic fluorescent compound having a structure represented by formula I-c is prepared according to “A new non-fullerene acceptor based on the combination of a heptacyclic benzothiadiazole unit and a thiophene-fused end group achieving over 13% efficiency” (Yunqiang Zhang, Fangfang Cai, Jun Yuan, Qingya Wei, Liuyang Zhou, Beibei Qiu, Yunbin Hu, Yongfang Li, Hongjian Peng and Yingping Zou., Phys. Chem. Chem. Phys., 2019, 21, 26557-26563 (incorporated by reference).

The present disclosure further provides use of an NIR-II organic fluorescent compound in non-diagnostic and non-therapeutic vascular fluorescence imaging, wherein the NIR-II organic fluorescent compound has a structure represented by formula I,

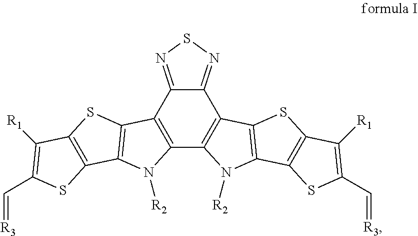

wherein in formula I,
R₁ and R₂ each represent branched or linear alkyl; and
R₃ represents

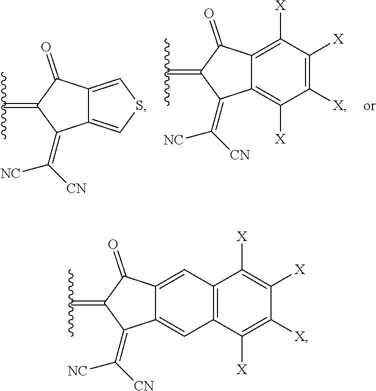

X being at least one selected from the group consisting of H, F, and Cl.

The specific embodiments of the NIR-II organic fluorescent compound having a structure represented by formula I are the same as described above, which will not be repeated herein.

The present disclosure also provides use of a nanoimaging agent in the preparation of a bioimaging contrast agent, wherein the nanoimaging agent includes an NIR-II organic fluorescent compound having a structure represented by formula I, and an organic encapsulation matrix covering a surface of the NIR-II organic fluorescent compound having the structure represented by formula I,

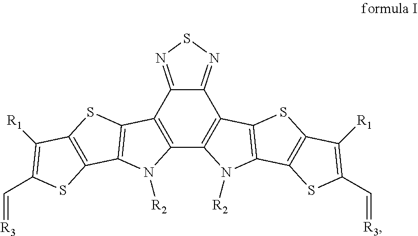

wherein in formula I,
R₁ and R₂ each represent branched or linear alkyl; and
R₃ represents

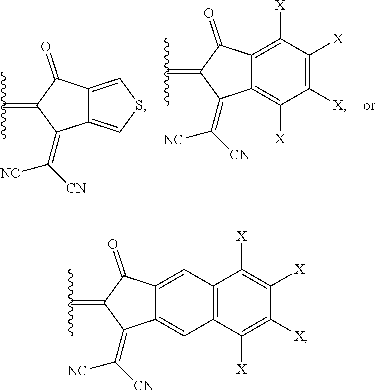

X in R₃ being at least one selected from the group consisting of H, F, and Cl.

The specific embodiments of the NIR-II organic fluorescent compound having a structure represented by formula I are the same as described above, which will not be repeated herein.

In some embodiments of the present disclosure, the bioimaging contrast agent is for monitoring vascular imaging in liver during ischemia-reperfusion, or monitoring vascular imaging during the kidney transplantation, or for vascular imaging in fluorescence imaging guided surgery.

In some embodiments of the present disclosure, the organic encapsulation matrix includes at least one of methoxypolyethylene glycol amine, 1,2-distearoyl-sn-glycero-3-phosphoethanolamine-poly(ethylene glycol), 1,2-distearoyl-sn-glycero-3-phosphoethanolamine-N-[maleimide(polyethylene glycol)], 1,2-distearoyl-sn-glycero-3-phosphoethanolamine-N-[folate(polyethylene glycol)], 1,2-distearoyl-sn-glycero-3-phosphoethanolamine-N-[thiol(polyethylene glycol)], 1,2-distearoyl-sn-glycero-3-phosphoethanolamine-N-[carboxy(polyethylene glycol)], 1,2-distearoyl-sn-glycero-3-phosphoethanolamine-N-[azido(polyethylene glycol)], 1,2-distearoyl-sn-glycero-3-ethanolamine-N-[biotin(polyethylene glycol)], 1-palmitoyl-2-oleoyl-sn-glycero-3-phosphoethanolamine, 1-stearoyl-2-oleoyl lecithin, 1,2-dipalmitoyl phosphoethanolamine-polyethylene glycol, polystyrene-grafted-poly(ethylene glycol), methoxy PEG polylactic acid-hydroxyacetic acid copolymer, and Pluronic® F-127.

In some embodiments of the present disclosure, the nanoimaging agent is prepared by a method including the steps of
mixing the organic encapsulation matrix, the NIR-II organic fluorescent compound having the structure represented by formula I, and an organic solvent to obtain a mixed solution; and mixing the mixed solution with water, and subjecting a resulting mixture to ultrasonic assembly, to obtain an assembly liquid;
introducing the assembly liquid into a dialysis bag and performing dialysis, to obtain a purified assembly material; and
concentrating the purified assembly material to a desired concentration, to obtain a solution of the nanoimaging agent.

In some embodiments of the present disclosure, the organic encapsulation matrix, the NIR-II organic fluorescent compound having the structure represented by formula I, and the organic solvent are mixed to obtain a mixed solution; the mixed solution is mixed with water, and a resulting mixture is subjected to ultrasonic assembly to obtain an assembly liquid. In some embodiments of the present disclosure, the organic solvent is tetrahydrofuran. In some embodiments, a mass ratio of the organic encapsulation matrix to the NIR-II organic fluorescent compound having the structure represented by formula I is in a range of 3:1 to 8:1, and preferably 4:1 to 7:1. In some embodiments, a volume ratio of the organic solvent to water is 1:10. There is no special requirement for the amount of the organic solvent in the present disclosure, as long as it is sufficient to ensure that the organic encapsulation matrix and the NIR-II organic fluorescent compound having the structure represented by formula I are completely dissolved. In some embodiments of the present disclosure, the ultrasonic assembly is performed under an ultrasonic power of 100-200 W, and preferably 150 W. In some embodiments, the ultrasonic assembly is performed for 3-10 min, and preferably 5 min.

In some embodiments, after obtaining the assembly liquid, the assembly liquid is introduced into a dialysis bag, and the dialysis is then performed to obtain a purified assembly material. In some embodiments of the present disclosure, the dialysis bag has a molecular weight cut-off of 3500. In some embodiments, the dialysis is performed by immersing the dialysis bag charged with the assembly liquid in water for dialysis. In some embodiments, the dialysis is performed at room temperature, and preferably for 48-72 h.

In some embodiments of the present disclosure, after obtaining the purified assembly material, the purified assembly material is concentrated to a desired concentration to obtain the solution of the nanoimaging agent. In some embodiments of the present disclosure, the concentration is performed by adsorbing water using polyethylene glycol. In some embodiments, the polyethylene glycol has an average molecular weight of 100,000. In some embodiments, a concentrated solution is obtained by concentrating, and the concentrated solution is filtered with a needle tube filter to filter off impurities, to obtain the solution of the nanoimaging agent.

The present disclosure provides use of a nanoimaging agent in non-diagnostic and non-therapeutic vascular fluorescence imaging, wherein the nanoimaging agent includes a second near-infrared (NIR-II) organic fluorescent compound having a structure represented by formula I, and an organic encapsulation matrix covering a surface of the NIR-II organic fluorescent compound having the structure represented by formula I,

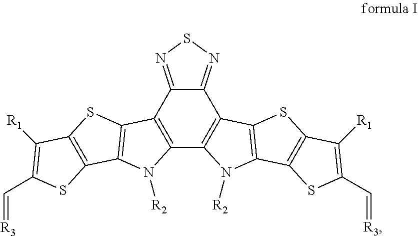

wherein in formula I,
R₁ and R₂ each represent branched or linear alkyl; and
R₃ represents

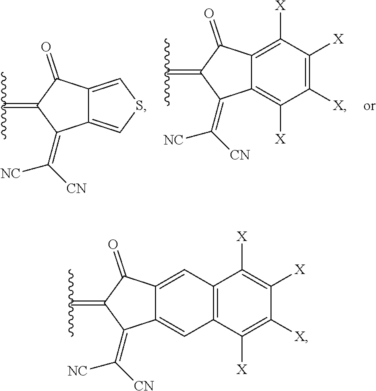

X in R₃ being at least one selected from the group consisting of H, F, and Cl.

The specific embodiments of the NIR-II organic fluorescent compound having the structure represented by formula I are the same as described above, which will not be repeated herein.

In specific examples of the present disclosure, specific embodiments of the use of the nanoimaging agent in non-diagnostic and non-therapeutic vascular fluorescence imaging include the following steps:

injecting the nanoimaging agent into a mouse through a tail vein and performing fluorescence imaging on an abdominal blood vessel under white light excitation.

In some embodiments of the present disclosure, the non-diagnostic and non-therapeutic vascular fluorescence imaging is in vivo imaging.

In specific examples of the present disclosure, the use in vascular fluorescence imaging includes monitoring vascular imaging in liver during ischemia-reperfusion, monitoring vascular imaging during the kidney transplantation, or vascular imaging during fluorescence image-guided surgery.

In specific examples of the present disclosure, specific embodiments of the use of the nanoimaging agent in monitoring the liver ischemia-reperfusion process for non-diagnostic and non-therapeutic purposes include the following steps:

injecting the nanoimaging agent into a liver ischemia-reperfusion model mouse through a tail vein, and performing fluorescence imaging on the mouse during the liver ischemia-reperfusion under white light excitation.

In specific examples of the present disclosure, the use of the nanoimaging agent in monitoring kidney transplantation process for non-diagnostic and non-therapeutic purpose includes the following steps:
injecting the nanoimaging agent into a renal transplant model of a New Zealand white rabbit through an ear vein, and performing fluorescence imaging on the white rabbit during kidney transplant under white light excitation.

In some embodiments of the present disclosure, in the use in vascular fluorescence imaging, a device for fluorescence imaging is an NIR-II small animal in vivo imaging device. In some embodiments, the vascular fluorescence imaging is performed under white light excitation, and the white light preferably has a wavelength of 400-800 nm. In some embodiments, a white light excitation source is an NIR-II small animal in vivo imaging device illumination lamp or a laparoscopic LED cold white light source, preferably having a wavelength of 400-800 nm.

In some embodiments of the present disclosure, the nanoimaging agent injected into the mouse during vascular fluorescence imaging or fluorescence imaging during liver ischemia-reperfusion has an effective concentration of not less than 300 μmol·L⁻¹, and a volume of not less than 100 μL.

In some embodiments of the present disclosure, the nanoimaging agent injected into a New Zealand white rabbit for fluorescence imaging during the kidney transplantation has an effective concentration of not less than 300 μmol·L⁻¹, and a volume of not less than 2 mL.

In order to further illustrate the present disclosure, the technical solutions according to the present disclosure will be described in detail in conjunction with examples, but they could not be construed as limiting the scope of the present disclosure.

### Example 1

The NIR-II organic fluorescent compound having a structure represented by formula I excited by white light used in this example was a compound of formula I-a (HY6), formula I-b (FY6), or formula I-c (Y6CT).

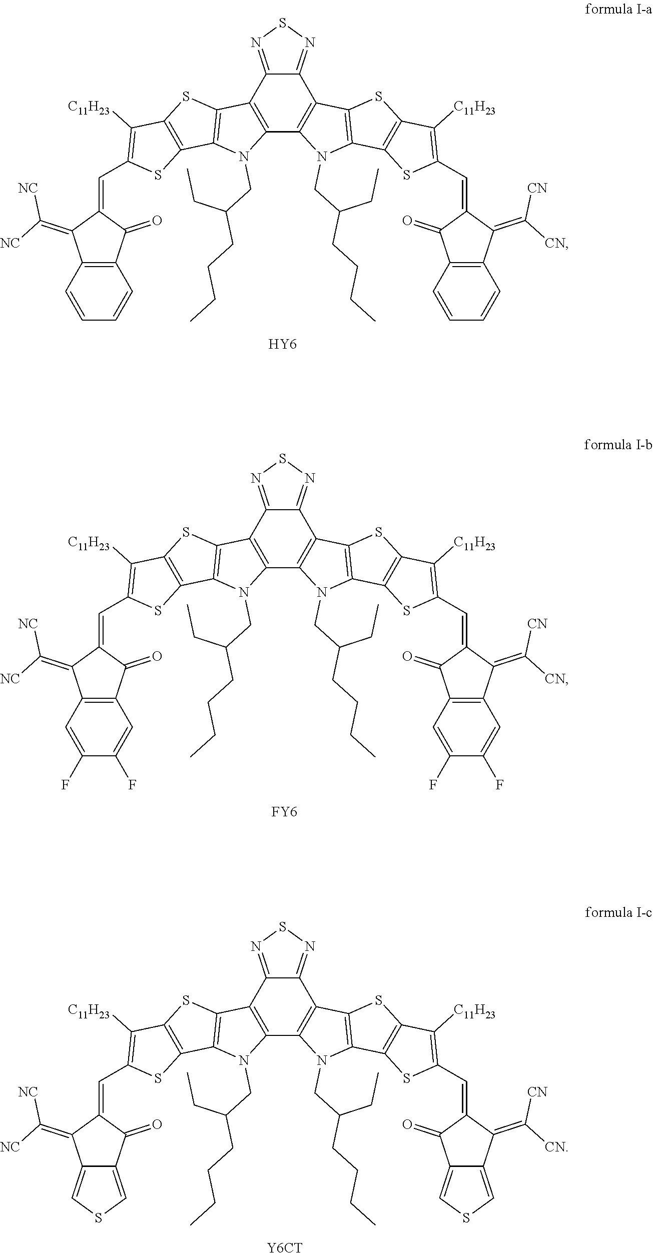

2 mg of the NIR-II organic fluorescent compound (HY6, FY6, or Y6CT) and 10 mg of DSPE-PEG₂₀₀₀ were dissolved in 1 mL of tetrahydrofuran, and 10 mL of deionized water was then added thereto and a resulting mixture was subjected to ultrasonic assembly. After that, a resulting nanoparticle solution was introduced into a dialysis bag with a molecular weight cut-off of 3500, and purified through dialysis for 72 h. The nanoparticle solution after dialysis was concentrated by adsorbing water using polyethylene glycol with an average molecular weight of 100,000. A resulting concentrated solution was then filtered with a needle tube filter to remove impurities, finally obtaining nanoimaging agents, namely HY6-NPs, FY6-NPs, and Y6CT-NPs. The effective concentrations of the nanoimaging agents were determined as 500 μmol·L⁻¹ through a pre-established concentration curve.

### Performance Tests:

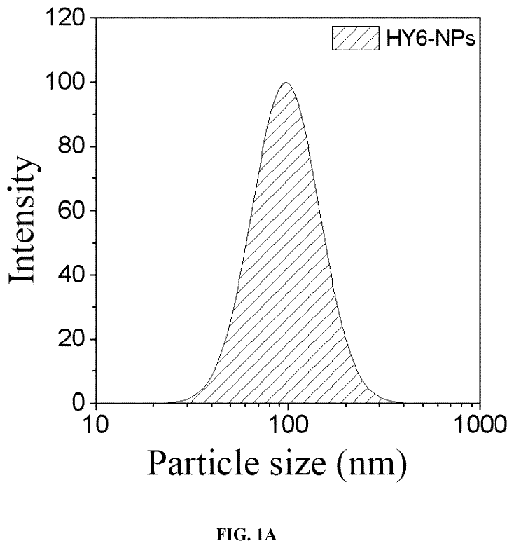

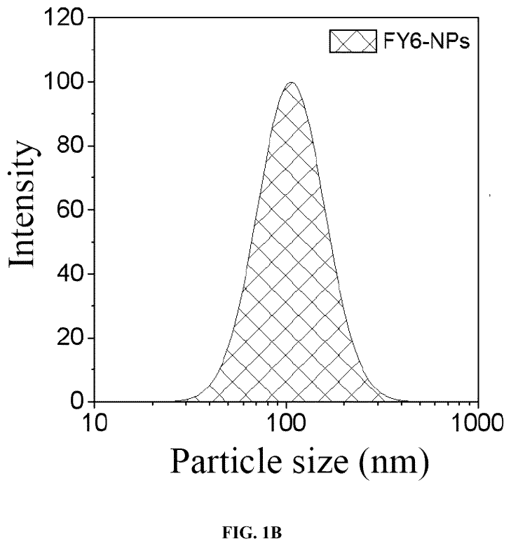

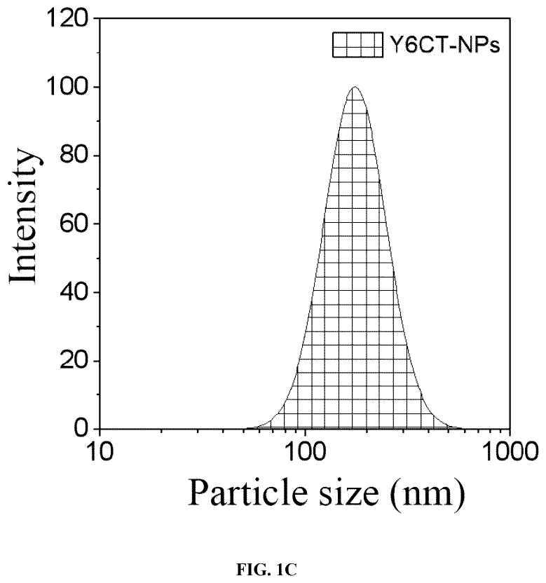

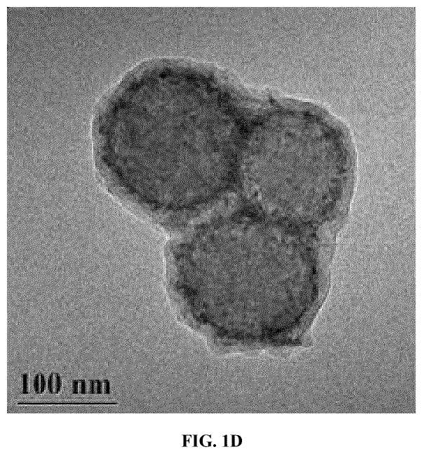

(1) Particle size test of the nanoimaging agents HY6-NPs, FY6-NPs, and Y6CT-NPs: 30 L of a nanoimaging agent was added to 3 mL of deionized water, and then the particle size thereof was measured using a dynamic light scattering instrument. The results are shown in FIG. 1A to FIG. 1D.

FIG. 1A to FIG. 1C show particle sizes of the nanoimaging agents HY6-NPs, FY6-NPs, and Y6CT-NPs, respectively. As can be seen from FIG. 1A to FIG. 1C, HY6-NPs, FY6-NPs, and Y6CT-NPs have a particle size of 95 nm, 104 nm, and 176 nm, respectively. From the TEM image in the inset, it can be further observed that the Y6CT-NPs have a spherical morphology with uniform size.

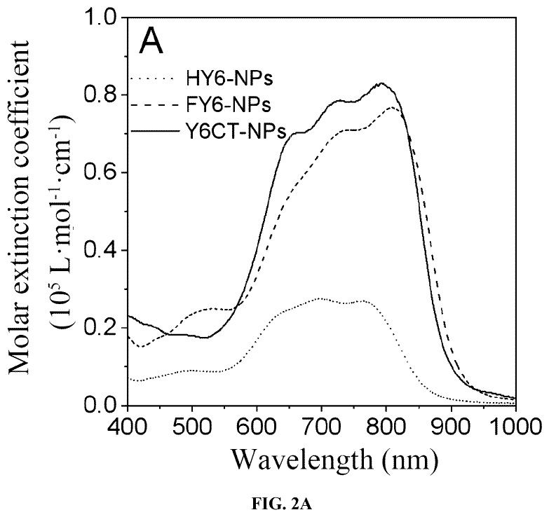

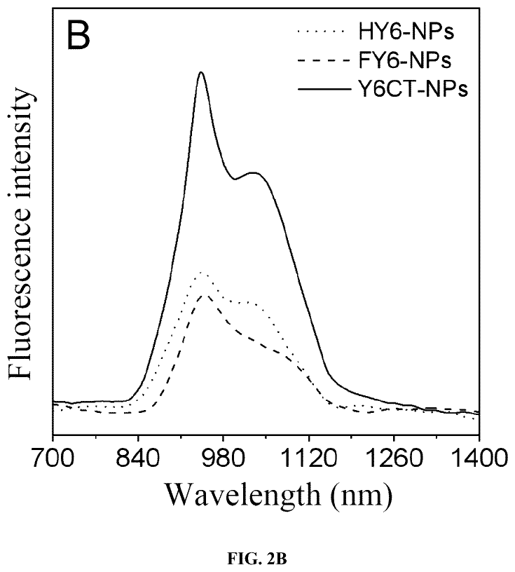

(2) Photophysical properties test of the nanoimaging agents HY6-NPs, FY6-NPs, and Y6CT-NPs: the absorption and emission spectra of Y6CT-NPs in aqueous solution were tested using an ultraviolet-visible-near infrared (UV-vis-NIR) spectrophotometer equipped with an integrating sphere module and a steady-state transient fluorescence spectrometer, respectively, at a test concentration of 10 μmol·L⁻¹. The results are shown in FIG. 2A and FIG. 2B.

FIG. 2A shows absorption spectra of the nanoimaging agents HY6-NPs, FY6-NPs, and Y6CT-NPs in aqueous solution. As can be seen from FIG. 2A, all the three nanoimaging agents exhibit strong absorption in the window of 400-1,000 nm. Among them, the molar extinction coefficients of HY6-NPs, FY6-NPs, and Y6CT-NPs at the maximum absorption peak are 2.68×10⁴ L·mol⁻¹·cm⁻¹ (763 nm), 7.76×10⁴ L·mol⁻¹·cm⁻¹ (811 nm), and 8.24×10⁴ L·mol⁻¹·cm⁻¹ (798 nm), respectively. Clearly, FY6-NPs and Y6CT-NPs exhibit higher absorptance and red-shifted absorption wavelengths compared with HY6-NPs, which is conducive to the absorption of white light energy. Under white light excitation, all the three nanoimaging agents exhibit fluorescence emission in the NIR-II region. In contrast, Y6CT-NPs exhibit the highest fluorescence intensity under white light (laparoscopic LED cold light source) excitation, with emission peaks at 947 nm and 1030 nm, which could be extended to 1,400 nm, being conducive to its use in white light-excited bioimaging.

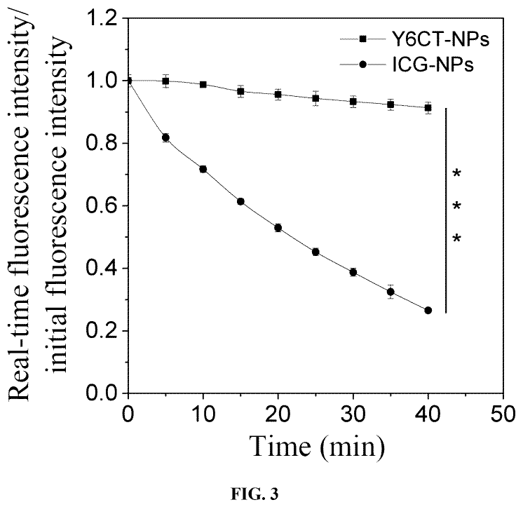

(3) Photostability test of the nanoimaging agent Y6CT-NPs: commercial imaging agent indocyanine green (ICG) was selected as a reference. Y6CT-NPs and ICG were irradiated continuously by 50 mW·cm⁻² white light (laparoscopic LED cold light source) for 40 min, and the fluorescence intensity of the two were recorded at different time points, a ratio of which to the initial fluorescence intensity was calculated to compare the attenuation degree of fluorescence intensity and photostability between the two. The results are shown in FIG. 3.

FIG. 3 shows a comparison of the photostability between the nanoimaging agent Y6CT-NPs and the commercial imaging agent indocyanine green (ICG). As can be seen from FIG. 3 that the fluorescence intensity of Y6CT-NPs slightly decreases after continuous irradiation for 40 min, while the fluorescence intensity of ICG decreases significantly, its fluorescence intensity at 40 min being only 30% of the initial fluorescence intensity, indicating that Y6CT-NPs have good photostability.

(3) Relative quantum yield test of the nanoimaging agent Y6CT-NPs: the NIR-II commercial fluorescent dye IR26 was selected as a reference to test the relative quantum yields of FY6-NPs, Y6CT-NPs, and HY6-NPs. IR26 needs to be prepared into a solution in dichloroethane (DCE). Firstly, the absorption spectra of FY6-NPs, Y6CT-NPs, HY6-NPs, and IR26-DCE were tested separately to determine their concentrations corresponding to absorbance values at 808 nm of 0.02, 0.04, 0.06, 0.08, and 0.10, respectively. The fluorescence spectra of the four at their corresponding concentrations were tested when excited at 808 nm. The fluorescence spectra with emission wavelengths ranging from 850 to 1,400 nm were integrated in area and used as the y-axis, and the absorbance values were used as the x-axis, and a linear regression analysis was performed to obtain the slope. The relative quantum yields of FY6-NPs, Y6CT-NPs, and HY6-NPs were calculated according to Equation 1.

$$Quantum {yield}_{NPs to be tested} = Quantum {yield}_{IR 26} \times \frac{{Slope}_{NPs to be tested}}{{Slope}_{IR 26}} \times {( \frac{Refractive index of water}{Refractive index of dichloroet hane} )}^{2} , \qquad Equation 1$$

wherein in Equation 1, the refractive index of water is 1.333, and the refractive index of dichloroethane is 1.4448.

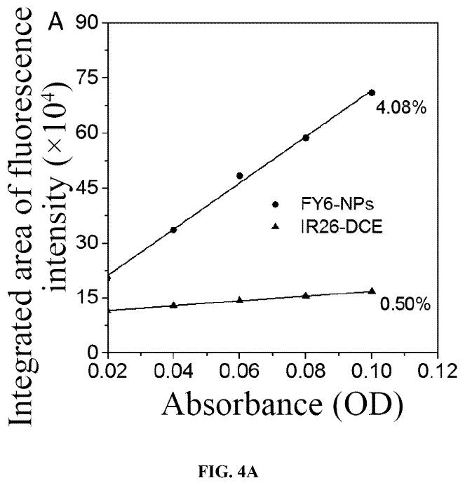

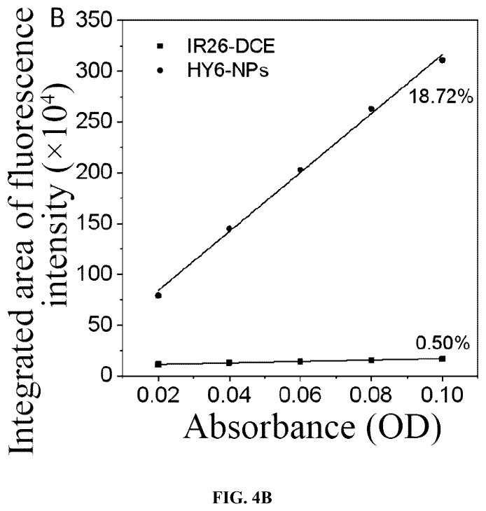

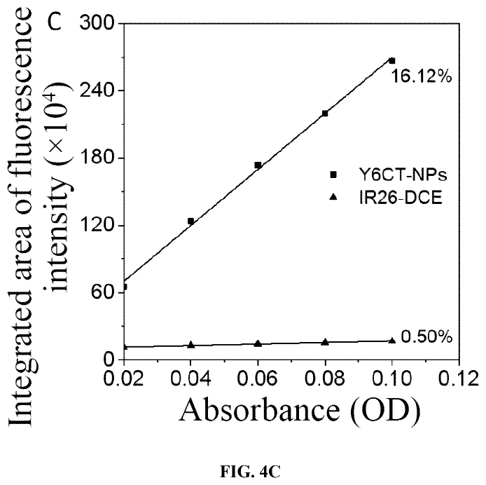

FIG. 4A to FIG. 4C show the relative quantum yield test results of the nanoimaging agents FY6-NPs (FIG. 4A), HY6-NPs (FIG. 4B), and Y6CT-NPs (FIG. 4C) with IR26 as a reference. As can be calculated from FIG. 4A to FIG. 4C, the relative quantum yields of FY6-NPs, Y6CT-NPs, and HY6-NPs are 4.08%, 18.72%, and 16.12%, respectively.

(4) Cytotoxicity test of nanoimaging agent Y6CT-NPs: cells at logarithmic growth stage were inoculated onto a 96-well plate at a density of 5×10³ cells/well, and incubated in a carbon dioxide incubator (37° C., 5% CO₂) for 24 hours. Subsequently, different concentrations of Y6CT-NPs were added thereto. After further incubation for 20 h, the cells were irradiated with white light (laparoscopic LED cold light source) (50 mW·cm⁻²) for 30 min (light irradiation group) or placed in dark for 30 min (dark group), and then further incubated for 4 h. After incubation, 10 μL of 3-(4,5-dimethylthiazole-2)-2,5-diphenyltetrazolium bromide (MTT) solution (5 mg·mL⁻¹) was added into each well, and a resulting system was left to stand for 4 h. The MTT solution was then removed, and 100 L of DMSO was added to each well. The absorbance of the product was measured using a microplate reader at a wavelength of 490 nm. The results were expressed as the percentage of live cells in the treated cells relative to the untreated control group cells. The relative cell viability was calculated according to Equation 2:

$$Cell viability ( \% ) = ( {OD}_{sample} - {OD}_{background} ) / ( {OD}_{control} - {OD}_{background} ) \times 1 \; 00 \% . \qquad Equation 2$$

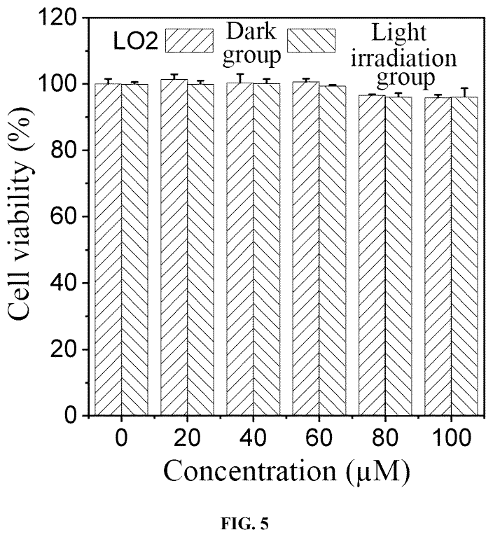

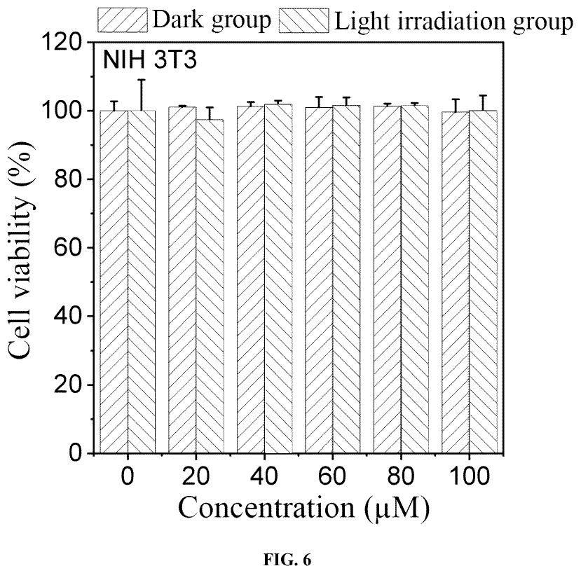

The results are shown in FIG. 5 and FIG. 6.

FIG. 5 shows the dark toxicity and phototoxicity of the nanoimaging agent Y6CT-NPs at different concentrations to human normal liver cells (LO2 cells), and FIG. 6 shows the dark toxicity and phototoxicity of the nanoimaging agent Y6CT-NPs at different concentrations to mouse embryonic fibroblasts (NIH 3T3 cells). As can be seen from FIG. 5 and FIG. 6, Y6CT-NPs have no obvious dark toxicity and phototoxicity on both LO2 cells and NIH 3T3 cells, indicating that Y6CT-NPs have minimal toxic side effects and good biocompatibility.

### Example 2

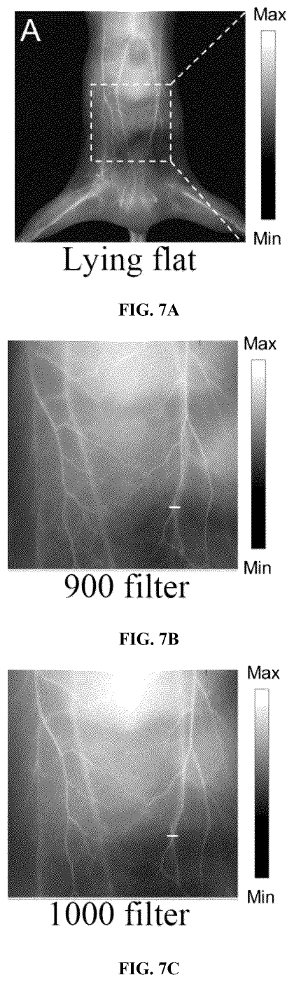

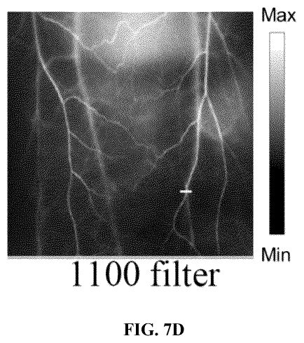

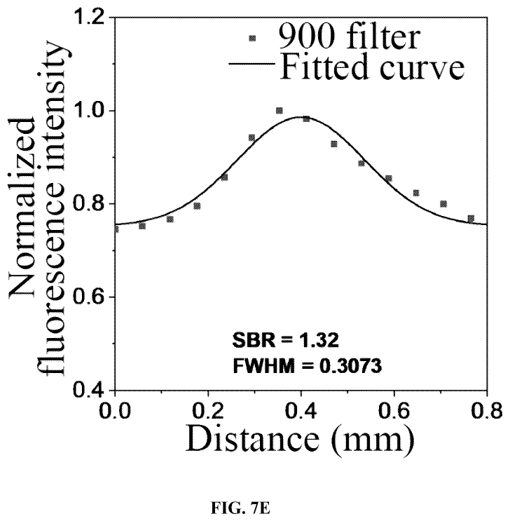

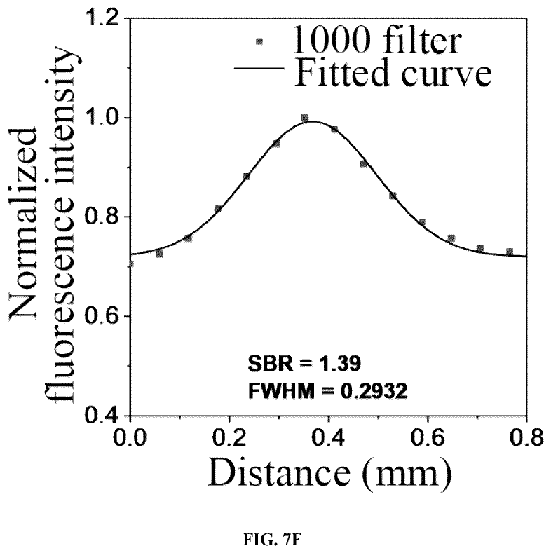

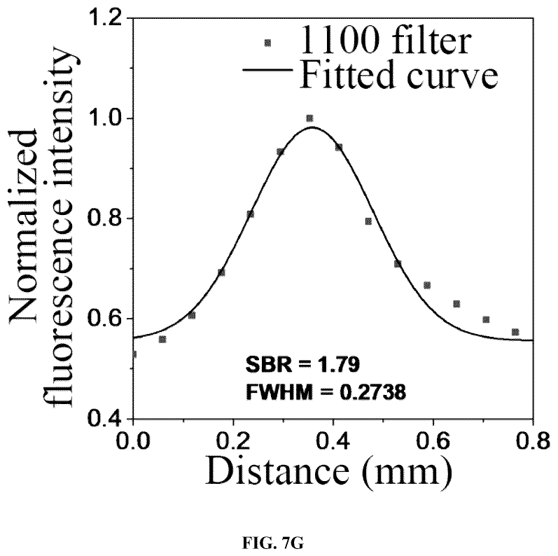

High-resolution NIR-II fluorescence imaging ability test of the nanoimaging agent Y6CT-NPs on abdominal blood vessels in a mouse: 100 μL of Y6CT-NPs with a concentration of 300 μmol·L⁻¹ was injected into a BALBC/b mouse through a tail vein, and the in vivo fluorescence of the mouse was imaged by an NIR-II small animal in vivo imaging instrument. The excitation light source was the illumination light source (16.5 mW·cm⁻²) of the NIR-II small animal in vivo imaging instrument. Fluorescence images were collected by adjusting filters of different wavelengths. The results are shown in FIG. 7A to FIG. 7D. The imaging resolutions under different filter conditions were analyzed using an imaging software. The results are shown in FIG. 7E, FIG. 7F, and FIG. 7G.

FIG. 7A to FIG. 7D show an NIR-II fluorescence image of abdominal blood vessels in a normal mouse under different filter conditions using the nanoimaging agent Y6CT-NPs. As can be seen from FIG. 7A to FIG. 7D, after tail-vein injection of Y6CT-NPs, the blood vessels of the mouse are quickly “lighted-up” and fluorescence signals appear in the NIR-II window. As adjusting the filter from 900 nm to 1,100 nm, the sharpness of the fluorescence image gradually improves. Under the condition of 1,100 nm filter, abdominal blood vessels are clearly visible and distinguished from surrounding tissues. FIG. 7E, FIG. 7F, and FIG. 7G show resolution analysis graphs under different filter conditions. As can be seen from FIG. 7E to FIG. 7G, as the filter conditions are gradually adjusted, the signal-to-background ratio (SBR) of the imaging gradually increases. Under the condition of 1100 nm filter (i.e., long pass filter), SBR reaches 1.79, and the half peak width decreases to 0.2738 mm. These results indicate that Y6CT-NPs have good high-resolution NIR-II fluorescence imaging ability for mouse abdominal blood vessels under white light excitation.

### Example 3

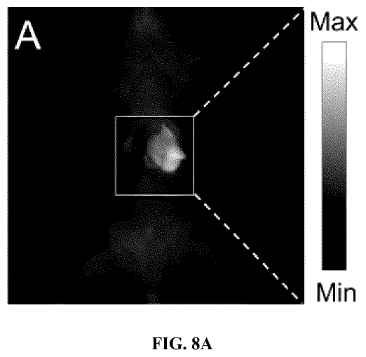

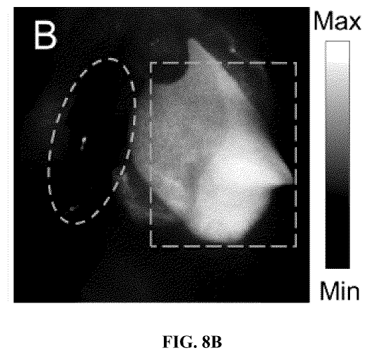

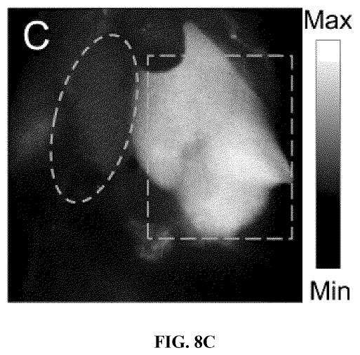

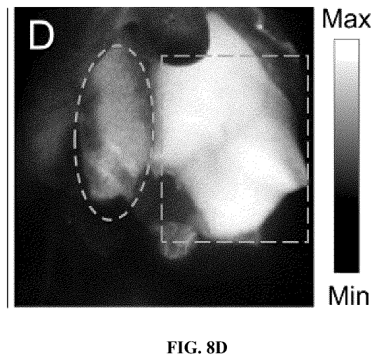

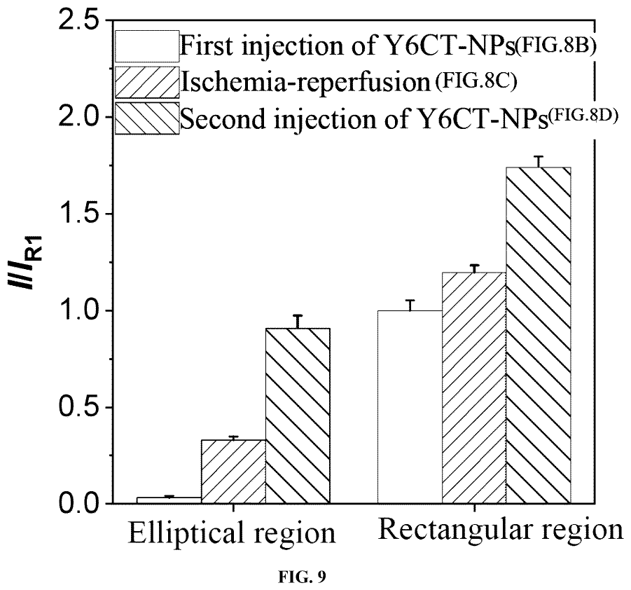

High-resolution NIR-II fluorescence imaging ability test of the nanoimaging agent Y6CT-NPs during mouse liver ischemia-reperfusion: 100 μL of Y6CT-NPs with a concentration of 300 μmol·L⁻¹ was injected into a BALBC/b mouse through a tail vein, and the in vivo fluorescence of the mouse liver was imaged by an NIR-II small animal in vivo imaging instrument. The excitation light source was the illumination light source (16.5 mW·cm⁻²) of the NIR-II small animal in vivo imaging instrument. The results are shown in FIG. 8A, FIG. 8B, FIG. 8C, and FIG. 8D. Fluorescence signal intensities of different regions were analyzed. The results are shown in FIG. 9.

FIG. 8A, FIG. 8B, FIG. 8C, and FIG. 8D show real-time NIR-II fluorescence images during mouse liver ischemia-reperfusion by using the nanoimaging agent Y6CT-NPs. FIG. 9 shows fluorescence intensity analysis of different regions during mouse liver ischemia-reperfusion monitored by using Y6CT-NPs. In conjunction with FIG. 8B and FIG. 9, it can be seen that, after tail-vein injection of Y6CT-NPs, a clear NIR-II fluorescence signal (fluorescence intensity recorded as I_{R1}) could be observed in the healthy liver region (rectangular region), while the fluorescence signal in the left lower lobe (ischemic part, elliptical region) could be negligible (0.03I_{R1}) due to the obstruction of the hepatic portal vein by hemostatic forceps. At 1 h after injection, the hemostatic forceps were removed, and the NIR-II fluorescence signal in the elliptical region gradually increased to 0.33I_{R1}, indicating that blood flow had been restored to the ischemic part (FIG. 8C). However, the fluorescence intensity of the elliptical region is much lower than that of the normal liver, which is related to the metabolism of Y6CT-NPs. After re-injection of Y6CT-NPs, the fluorescence intensity of the elliptical region rapidly increased from 0.33I_{R1} to 090I_{R1}, and the change was similar to the fluorescence signal change in healthy liver (from 1.19I_{R1} to 174I_{R1}), further proving that the blood supply function of liver had been restored. These results fully demonstrate that Y6CT-NPs could achieve real-time high-resolution NIR-II fluorescence imaging during mouse liver ischemia-reperfusion under white light excitation.

### Example 4

High-resolution NIR-II fluorescence imaging ability test of the nanoimaging agent Y6CT-NPs during the kidney transplantation in a New Zealand white rabbit: the real-time fluorescence monitoring ability test of Y6CT-NPs during the kidney transplantation was divided into three parts: fluorescence imaging ability test on blood vessels in the donor kidney region, real-time monitoring of renal blood vessel anastomosis during the kidney transplantation, and real-time monitoring of blood supply to ureter of the transplanted kidney. Models were established separately to perform fluorescence imaging during these processes.

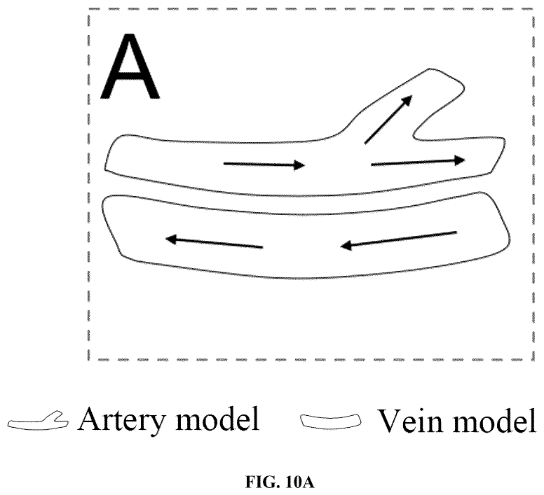

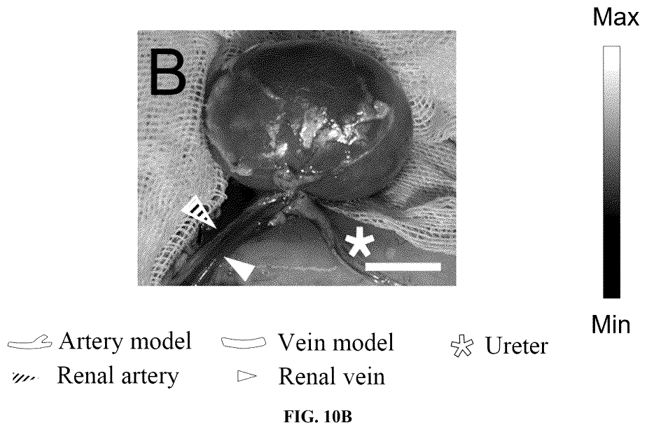

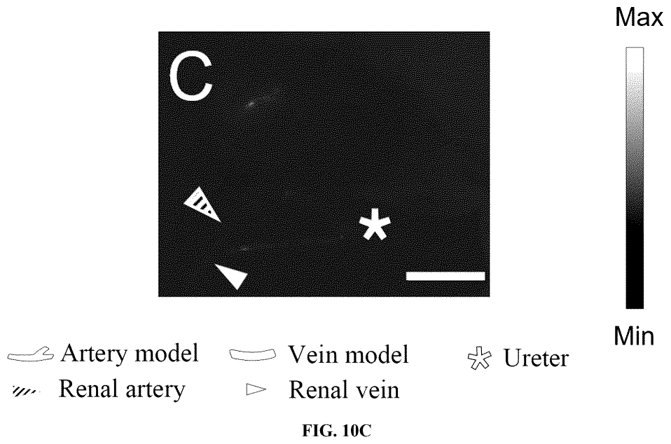

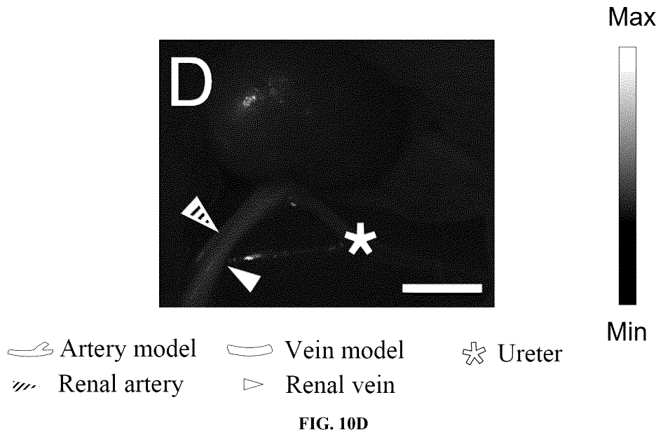

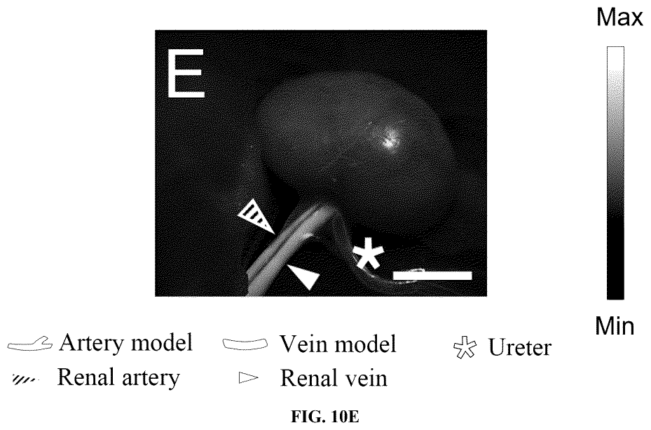

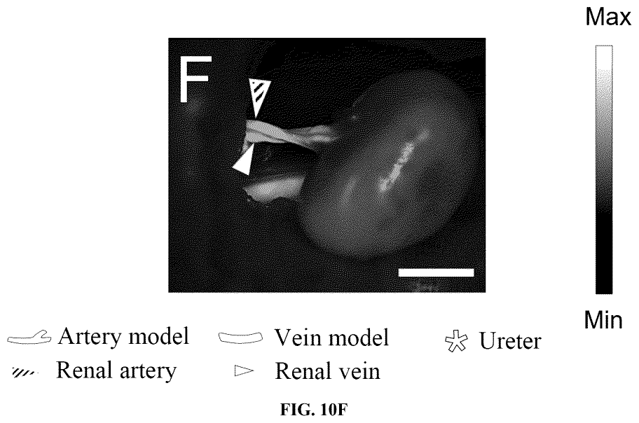

(1) Real-time NIR-II fluorescence imaging ability test of the nanoimaging agent Y6CT-NPs on the donor kidney region in a New Zealand white rabbit: 2 mL of Y6CT-NPs with a concentration of 300 μmol·L⁻¹ was injected through an ear vein into the New Zealand white rabbit, and the NIR-II fluorescence of the donor kidney region in the New Zealand white rabbit was imaged by an NIR-II small animal in vivo imaging instrument. The excitation light source was a laparoscopic LED cold white light source (20 mW·cm⁻²). The results are shown in FIG. 10A to FIG. 10F.

FIG. 10A to FIG. 10F show a real-time NIR-II fluorescence image of the donor kidney region in the New Zealand white rabbit using the nanoimaging agent Y6CT-NPs. As can be seen from FIG. 10C and FIG. 10D, as the nanoimaging agent Y6CT-NPs is injected, the donor renal artery (hollow triangular arrow) and vein (patterned triangular arrow) are immediately “lighted up”, resulting in clear NIR-II fluorescence signals. As time goes on, the boundary of renal artery and vein gradually becomes clear, and fluorescence intensity of the kidney gradually increases (FIG. 10E). When the kidney is rotated counterclockwise 180° around the renal artery and vein, the boundary of the renal artery and vein remains clear and is easy to identify (FIG. 10F). These results fully demonstrate that Y6CT-NPs could achieve high-resolution NIR-II fluorescence imaging of renal artery and vein in different orientations under white light excitation.

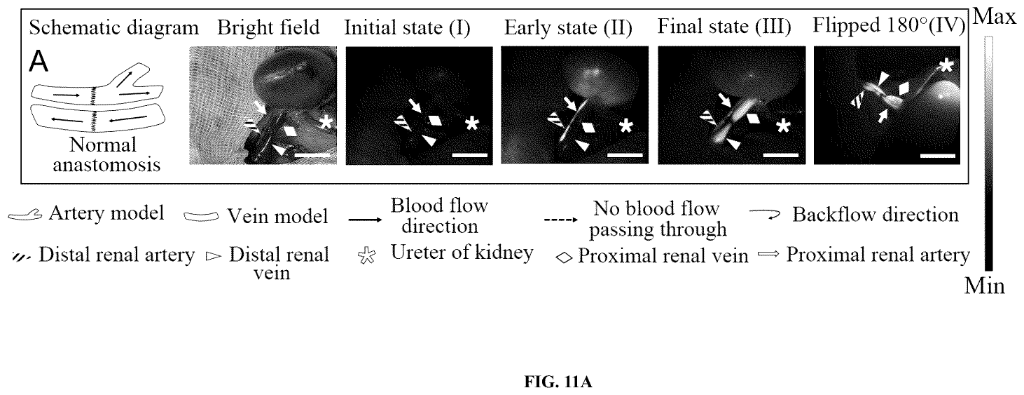

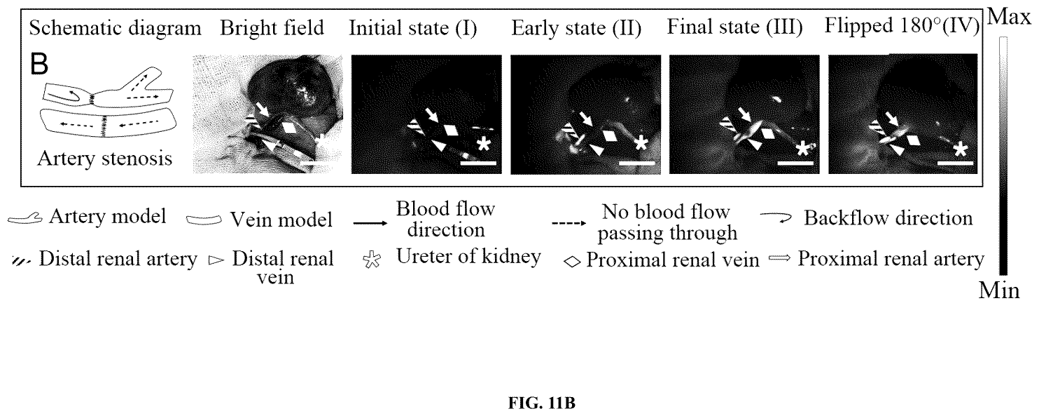

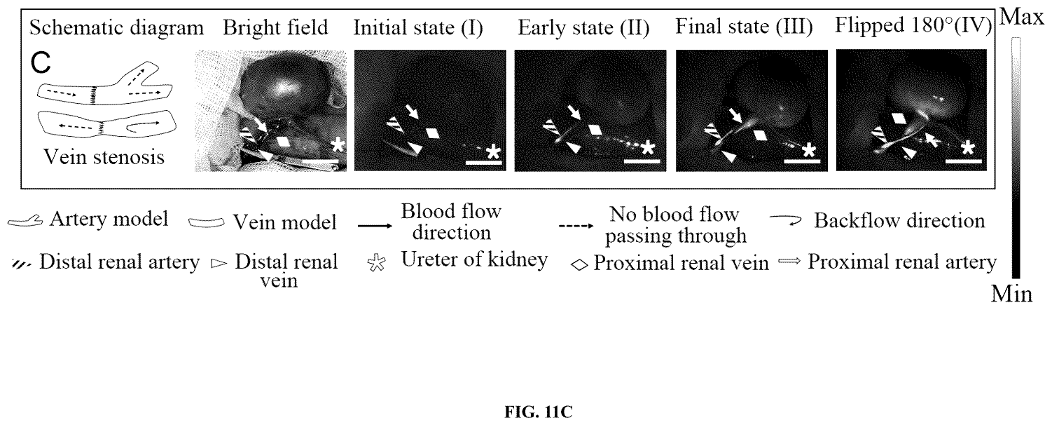

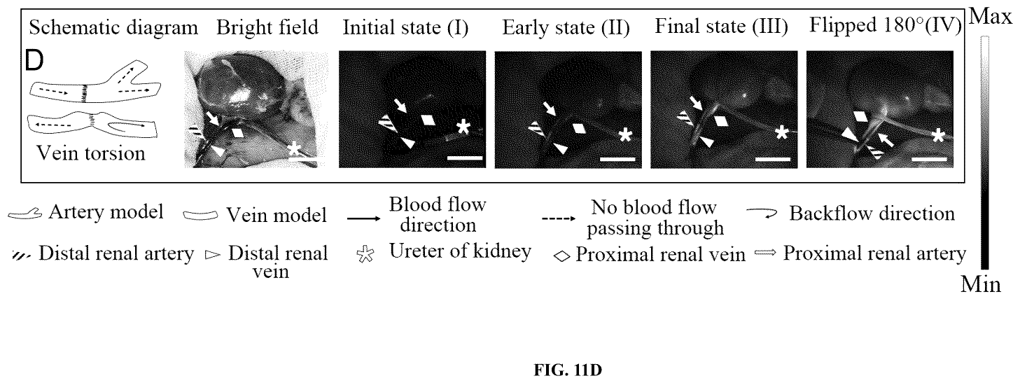

(2) Real-time monitoring ability test of the nanoimaging agent Y6CT-NPs on renal blood vessel anastomosis during the kidney transplantation: whether the renal blood vessels are anastomosed during the kidney transplantation determines whether the kidney transplantation is successful. Therefore, monitoring the renal blood vessel anastomosis during the kidney transplantation is very important. Four models were established to evaluate the real-time monitoring ability of Y6CT-NPs on renal blood vessel anastomosis during the kidney transplantation, namely, normal anastomosis of transplanted renal blood vessels, anastomotic stenosis of transplanted renal artery (complete occlusion), anastomotic stenosis of transplanted renal vein, and anastomotic torsion of transplanted renal vein. The results are shown in FIG. 11A to FIG. 11D.

### (i) Normal Anastomosis of Transplanted Renal Blood Vessels

A normal anastomosis model of transplanted renal blood vessels was constructed by anastomosing the donor renal artery and vein and acceptor renal artery and vein using polyglactin suture. 2 mL of Y6CT-NPs with a concentration of 300 μmol/L was injected through an ear vein to a New Zealand white rabbit, and the NIR-II fluorescence of the transplanted kidney region in the New Zealand white rabbit was imaged by an NIR-II small animal in vivo imaging instrument. The excitation light source was a laparoscopic white light source (20 mW·cm⁻²). The results are shown in FIG. 11A.

FIG. 11A shows a real-time NIR-II fluorescence image of the transplanted kidney region with normal anastomosis of renal blood vessels using the nanoimaging agent Y6CT-NPs. As can be seen from FIG. 11A, after injection of the nanoimaging agent Y6CT-NPs, when hemostatic clips applied to artery and vein are released, the transplanted renal artery is first “lighted up” ((II) of FIG. 11A, hollow triangular arrow), showing clear NIR-II fluorescence signals. Subsequently, NIR-II fluorescence is also observed in kidney and renal vein ((III) of FIG. 11A, patterned triangular arrow). When the kidney is rotated counterclockwise 180° around the renal artery and vein, the outlines of the renal artery and vein are still clearly visible. The changes in these NIR-II fluorescence signals indicate that the blood vessel anastomosis of the transplanted kidney is normal and there is no blood leakage at the blood vessel anastomosis site. The transplanted renal blood vessels are unobstructed, and no stenosis is found at the vessel anastomosis site.

### (ii) Anastomotic Stenosis of Transplanted Renal Artery (Complete Occlusion)

An anastomotic stenosis model (complete occlusion) of transplanted renal artery was constructed by anastomosing the donor renal artery and vein and acceptor renal artery and vein using polyglactin suture. 2 mL of Y6CT-NPs with a concentration of 300 μmol/L was injected through an ear vein into a New Zealand white rabbit, and the NIR-II fluorescence of the transplanted kidney region in the New Zealand white rabbit was imaged by an NIR-II small animal in vivo imaging instrument. The excitation light source was a laparoscopic white light source (20 mW·cm⁻²). The results are shown in FIG. 11B.

FIG. 11B shows the real-time NIR-II fluorescence image of the transplanted kidney region with anastomotic stenosis (complete occlusion) of renal artery using the nanoimaging agent Y6CT-NPs. As can be seen from FIG. 11B, after injection of the nanoimaging agent Y6CT-NPs, when hemostatic clips applied to artery and vein are released, only the distal renal artery ((II) of FIG. 11B, hollow triangular arrow) is “lighted up” by NIR-II fluorescence, while the fluorescence signal is not observed in the transplanted renal artery ((II) of FIG. 11B, directional arrow) due to anastomotic stenosis of the renal artery, indicating that the arterial stenosis blocks blood from flowing to the kidney. Subsequently, blood flow at the stenosed anastomosis site of the transplanted renal artery is opened by adjusting the positions of the transplanted renal blood vessels, but only the proximal transplanted renal artery emits weak fluorescence ((IV) of FIG. 11B, directional arrow), indicating complete occlusion of the renal artery. In addition, the transplanted renal vein displays an NIR-II fluorescence signal ((III) of FIG. 11B, patterned triangular arrow), which is caused by inferior vena cava backflow. These changes in NIR-II fluorescence signals indicate stenosis at the anastomotic site of the transplanted renal artery, resulting in complete occlusion of the arterial vessels.

(iii) Anastomotic Stenosis of Transplanted Renal Vein

An anastomotic stenosis model of transplanted renal vein was constructed by anastomosing the donor renal artery and vein and acceptor renal artery and vein using polyglactin suture. 2 mL of Y6CT-NPs with a concentration of 300 μmol·L⁻¹ was injected through an ear vein into a New Zealand white rabbit, and the NIR-II fluorescence of the transplanted kidney region in the New Zealand white rabbit was imaged by an NIR-II small animal in vivo imaging instrument. The excitation light source was a laparoscopic white light source (20 mW·cm⁻²). The results are shown in FIG. 11C.

FIG. 11C shows the real-time NIR-II fluorescence image of the transplanted kidney region with anastomotic stenosis of renal vein using the nanoimaging agent Y6CT-NPs. As can be seen from FIG. 11C, after injection of the nanoimaging agent Y6CT-NPs, when hemostatic clips applied to artery and vein are released, the distal transplanted renal artery first emits fluorescence ((II) of FIG. 11C, hollow triangular arrow). Subsequently, the proximal transplanted renal artery emits fluorescence ((III) of FIG. 11C, directional arrow). Finally, the transplanted kidney emits weak fluorescence. Due to anastomotic stenosis of the transplanted renal vein, it is difficult for the blood flow to pass through the anastomotic site of the transplanted renal vein and flow back to the inferior vena cava. As such, at the early stage, fluorescence signals do not appear in the transplanted renal vein ((II) of FIG. 11C, patterned triangular arrows, and hollow triangular arrows). Subsequently, by adjusting the transplanted renal blood vessels, the blood flow at the stenosed anastomosis site of the transplanted renal vein is opened, as shown in (III) of FIG. 11C. Blood flow flows out of the transplanted kidney, slowly passes through the anastomotic site of vein, and flows back to the distal end of the transplanted renal vein and inferior vena cava. The transplanted renal vein emits weak fluorescence ((III) of FIG. 11C, directional arrow), and the NIR-II fluorescence of the transplanted kidney is slightly enhanced. Subsequently, the kidney is rotated counterclockwise 180° around the renal artery and vein, and the transplanted kidney and the transplanted renal artery and vein display consistent fluorescence signals ((IV) of FIG. 11C). These changes in NIR-II fluorescence signals indicate that the anastomotic site of the transplanted renal vein is partially occluded, not completely occluded. After adjusting the renal blood vessels, part of transplanted renal vein is reopened, and the blood perfusion of the transplanted kidney is increased than before, which however has not yet reached a normal state.

### (iv) Anastomotic Torsion of Transplanted Renal Vein

An anastomotic torsion model of transplanted renal vein was constructed by anastomosing the donor renal artery and vein and acceptor renal artery and vein using polyglactin suture. 2 mL of Y6CT-NPs with a concentration of 300 μmol·L⁻¹ was injected through an ear vein into a New Zealand white rabbit, and the NIR-II fluorescence of the transplanted kidney region in the New Zealand white rabbit was imaged by an NIR-II small animal in vivo imaging instrument. The excitation light source was a laparoscopic white light source (20 mW·cm⁻²). The results are shown in FIG. 11D.

FIG. 11D shows the real-time NIR-II fluorescence image of the transplanted kidney region with torqued anastomotic site of transplanted renal vein using the nanoimaging agent Y6CT-NPs. As can be seen from FIG. 11D, after injection of the nanoimaging agent Y6CT-NPs, when hemostatic clips applied to artery and vein are released, the distal transplanted renal artery first emits fluorescence signals ((II) of FIG. 11D, hollow triangular arrow), and subsequently the proximal transplanted renal artery emits fluorescence ((II) of FIG. 11D, directional arrow), and finally, the transplanted kidney emits weak fluorescence. Due to anastomotic torsion of the transplanted renal vein, it is difficult for the blood flow to pass through the anastomotic site of the transplanted renal vein and flow back to the inferior vena cava. As such, at the early stage, fluorescence signals do not appear in the transplanted renal vein ((II) of FIG. 11D, patterned triangular arrow and hollow rhombus arrow). Subsequently, by adjusting the transplanted renal blood vessels, the blood flow at the stenosed anastomosis site of the transplanted renal vein is opened, as shown in III of FIG. 11D; the blood flow flows out of the transplanted kidney, quickly passes through anastomotic site of vein, and flows back to the distal end of the transplanted renal vein and inferior vena cava. The transplanted renal vein emits fluorescence ((III) of FIG. 11D, directional arrow), and the NIR-II fluorescence of the transplanted kidney is enhanced to normal state. Subsequently, the kidney is rotated clockwise 180° around the renal artery and vein, and both the transplanted kidney and the transplanted renal artery and vein display consistent fluorescence signals ((IV) of FIG. 11D). The changes in these NIR-II fluorescence signals indicate that the anastomotic site of the transplanted renal vein is torqued. After adjusting directions of the transplanted renal blood vessels, the transplanted renal vein is reopened and the blood perfusion of the transplanted kidney is restored to a normal state.

By establishing models of different anastomosis states of renal blood vessels during the kidney transplantation, the real-time monitoring ability of the nanoimaging agent Y6CT-NPs was evaluated. The above experimental results fully demonstrate that Y6CT-NPs could achieve real-time high-resolution NIR-II fluorescence imaging monitoring of different anastomosis states of renal blood vessels during the kidney transplantation under white light excitation.

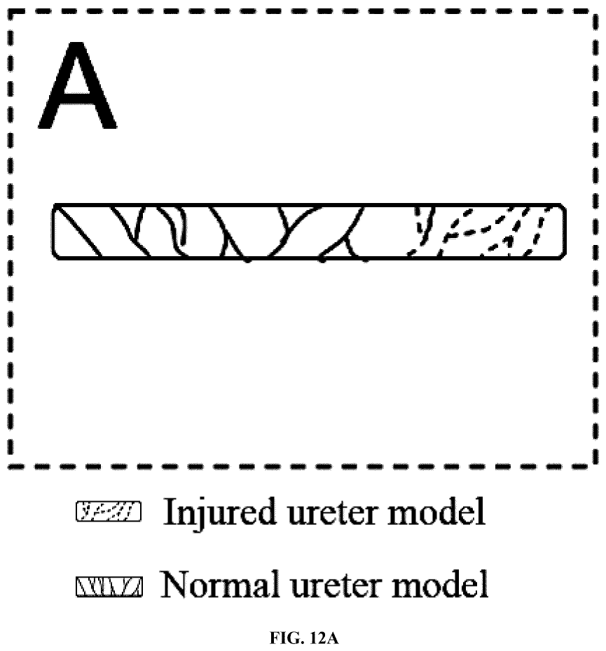

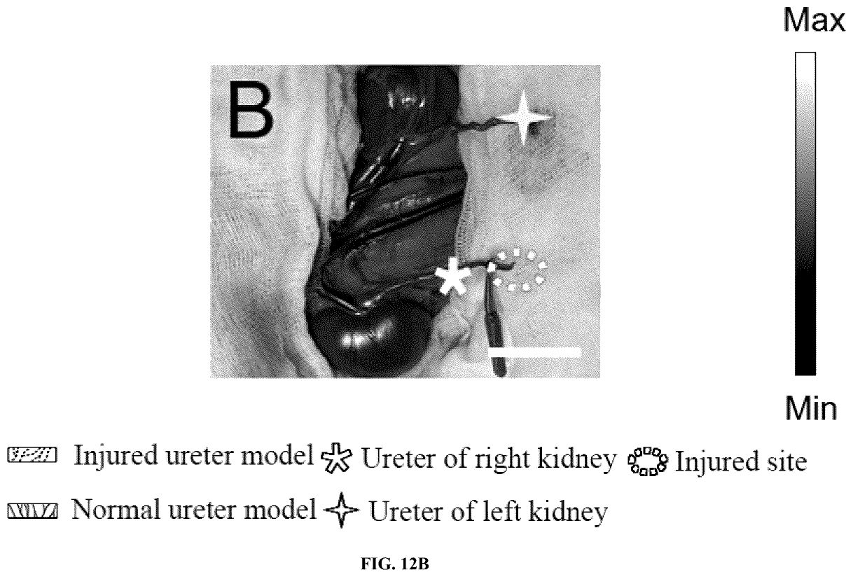

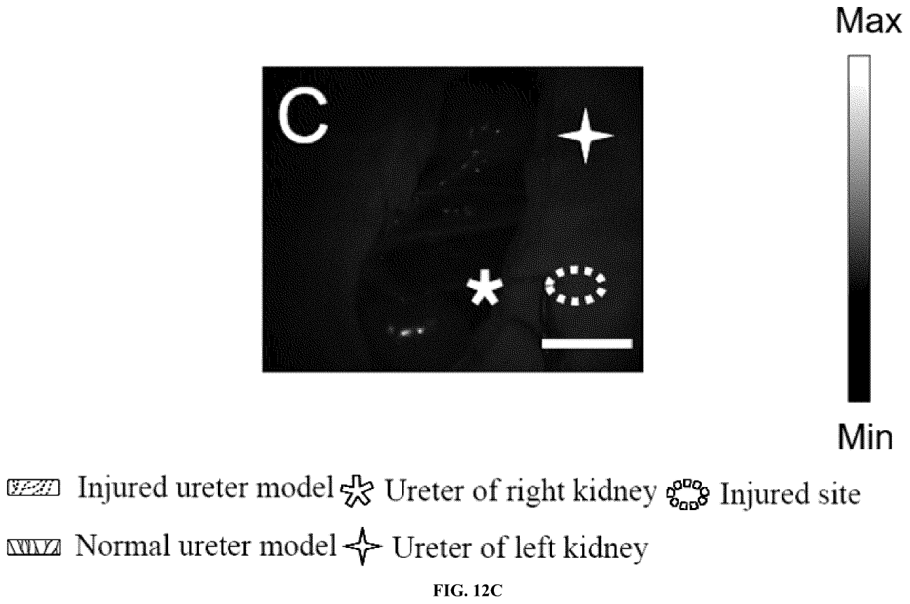

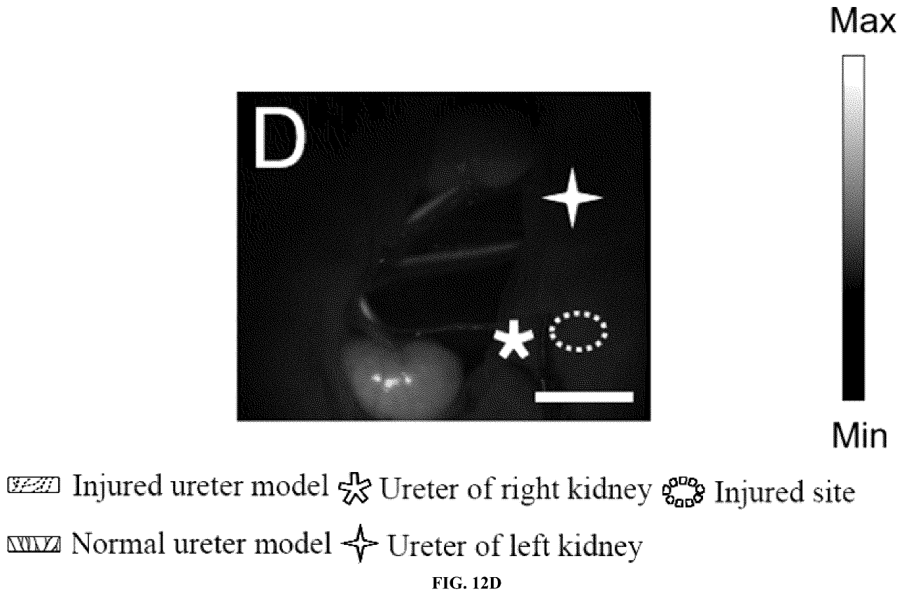

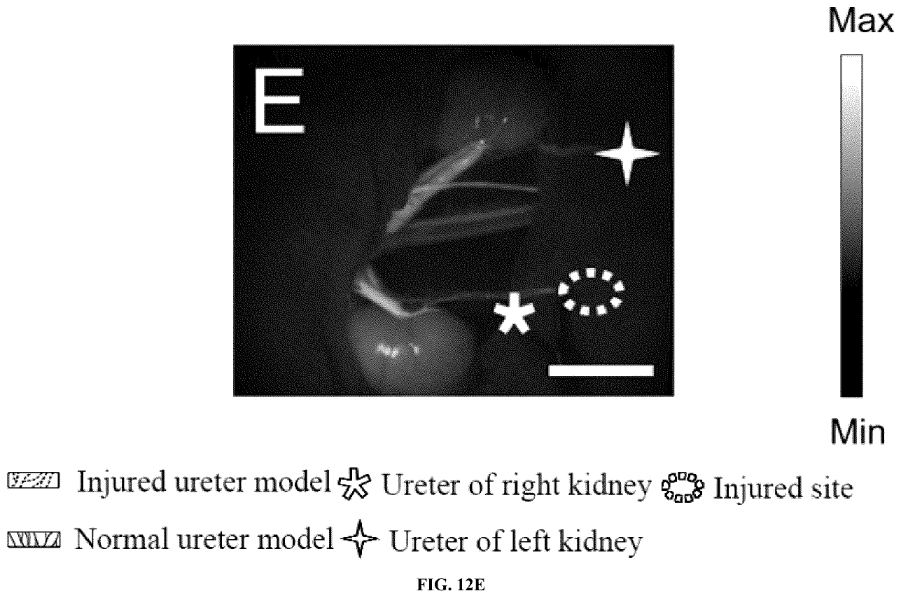

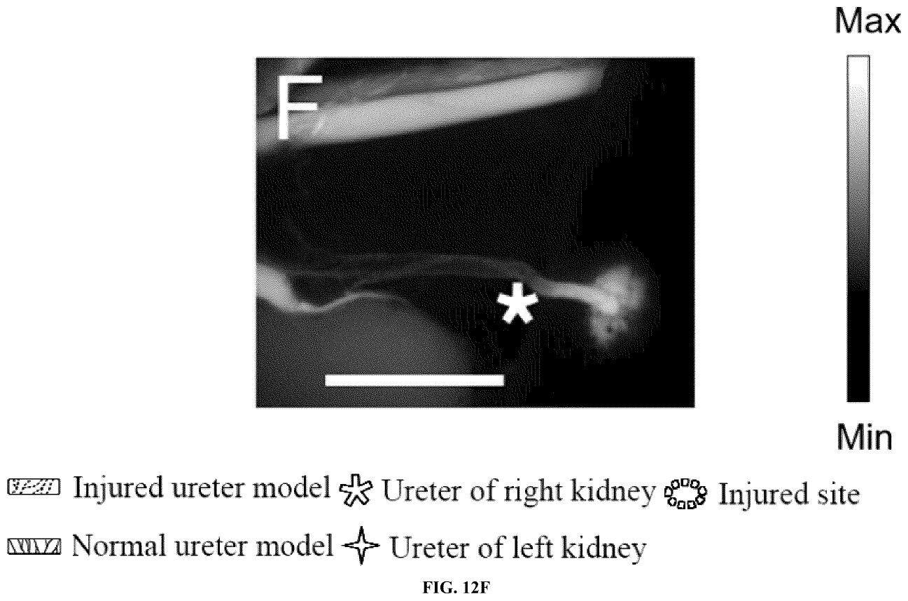

(3) Monitoring ability test of the nanoimaging agent Y6CT-NPs on blood supply to ureter of transplanted kidney: with the weak blood supply to ureter of transplanted kidney, if there is poor or even zero blood supply to terminal ureter of the transplanted kidney, it could lead to non healing of anastomosis site between ureter of the transplanted kidney and bladder, resulting in that urine flows into pelvic cavity and causes infection, and in severe cases, patient death. Therefore, the real-time monitoring of the blood supply to ureter of the transplanted kidney is crucial. Two models of normal ureter and injured ureter of transplanted kidney were established to evaluate the real-time monitoring ability of Y6CT-NPs on blood supply to ureter of transplanted kidney. 2 mL of Y6CT-NPs with a concentration of 300 μmol/L was injected through an ear vein to a New Zealand white rabbit, and the NIR-II fluorescence of the donor kidney region in the New Zealand white rabbit was imaged by an NIR-II small animal in vivo imaging instrument. The excitation light source was a laparoscopic white light source (20 mW·cm⁻²). The results are shown in FIG. 12A to FIG. 12F.

FIG. 12A to FIG. 12F show a real-time monitoring image of blood supply to ureter of the transplanted kidney during the kidney transplantation in a New Zealand white rabbit using the nanoimaging agent Y6CT-NPs. As can be seen from FIG. 12A to FIG. 12F, after injection of the nanoimaging agent Y6CT-NPs, fluorescence signals rapidly appear in kidneys at both sides and the artery and vein, and subsequently fluorescence signals appear in ureters at both sides (FIG. 12D), wherein the right renal ureter is damaged, while the left renal ureter is normal. It can be seen that, the fluorescence signal in ureter of the left kidney is normal (FIG. 12D, quadrangular asterisk), while in the right kidney, due to the presence of the vascular clamp, the fluorescence signal only appears at the proximal end of ureter but not at the distal end thereof (FIG. 12D, asterisk), indicating that there is no blood supply to the distal end (distanced from the vascular clamp applied) of ureter in the right kidney. After releasing the hemostatic clip, the blood supply to the distal end (distanced from the vascular clamp applied) of ureter of the right kidney does not recover, which is because the long-term ischemia leads to necrosis of blood vessels in this part of the ureter. The necrotic part is trimmed off, and blood supply to the terminal end of ureter in the right kidney is recovered. These results fully demonstrate that Y6CT-NPs could achieve real-time high-resolution NIR-II fluorescence imaging monitoring of blood supply to ureter in transplanted kidney under white light excitation.

Through the test of different model experiments, it is demonstrated that Y6CT-NPs could achieve high-resolution NIR-II fluorescence imaging during the kidney transplantation in a New Zealand white rabbit.

The test results of compounds HY6-NPs and FY6-NPs are substantially consistent with those of compound Y6CT-NPs mentioned above.

From the above examples, it can be seen that, the NIR-II organic nanoimaging agent for vascular imaging under white light excitation according to the present disclosure has a simple preparation method, has excellent luminescent properties, and could achieve high-resolution NIR-II imaging of mouse blood vessels under white light excitation, with good practical effects.

Although the above examples provide a detailed description of the present disclosure, they are only a part of the examples, not all of them. Other examples could also be obtained without creative labor based on the present examples, all of which shall fall within the scope of the present disclosure.

What is claimed is:

1. A method for preparing a bioimaging contrast agent, comprising using a second near-infrared organic fluorescent compound having a structure represented by formula I, 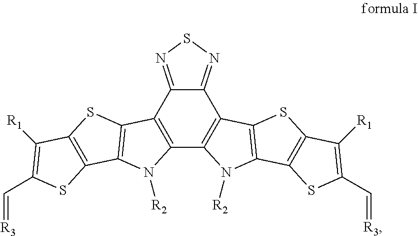
wherein in formula I,
R₁ and R₂ each represent branched or linear alkyl; and
R₃ represents 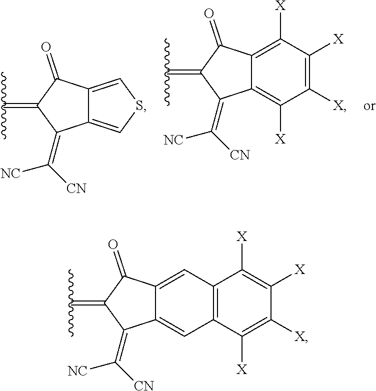
X being at least one selected from the group consisting of H, F, and Cl.

2. The method of claim 1, wherein R₁ and R₂ each represent C₁-C₁₁ branched or linear alkyl.

3. The method of claim 1, wherein R₃ represents 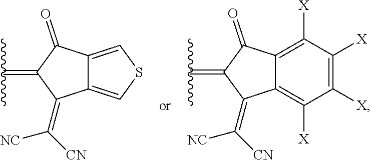
X being H, or X being H and F.

4. The method of claim 1, wherein the NIR-II organic fluorescent compound has a structure represented by formula I-a, formula I-b, or formula I-c, 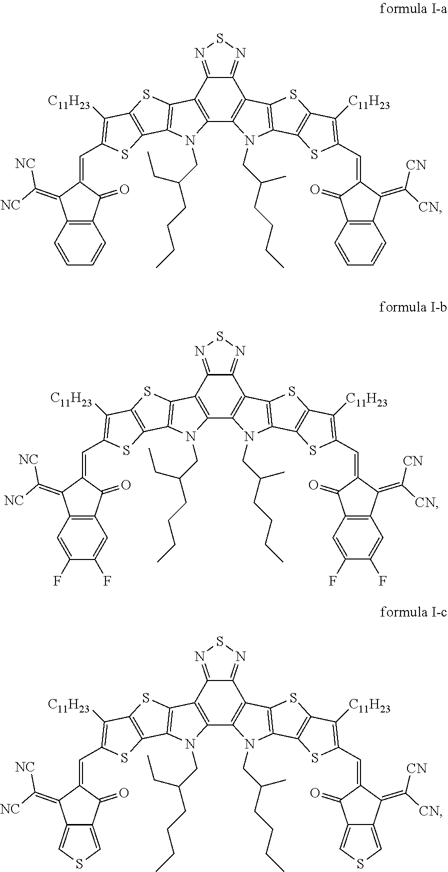
wherein in formula I-a, formula I-b, or formula I-c, C₁₁H₂₃ is linear alkyl.

5. A method for providing vascular fluorescence imaging, comprising using a second near-infrared organic fluorescent compound having a structure represented by formula I, 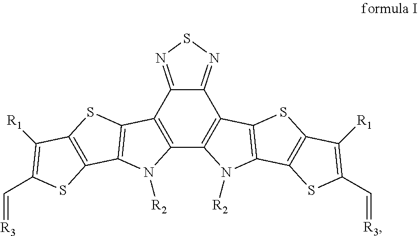
wherein in formula I,
R₁ and R₂ each represent branched or linear alkyl; and
R₃ represents 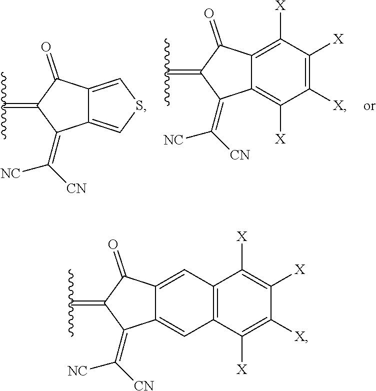
X being at least one selected from the group consisting of H, F, and Cl.

6. A nanoimaging agent, comprising a second near-infrared (NIR-II) organic fluorescent compound having a structure represented by formula I, and an organic encapsulation matrix covering a surface of the NIR-II organic fluorescent compound having the structure represented by formula I, 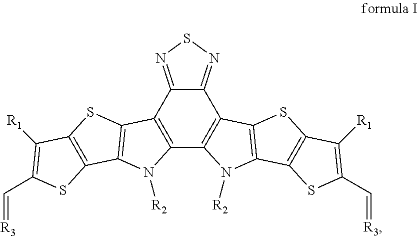
wherein in formula I,
R₁ and R₂ each represent branched or linear alkyl; and
R₃ represents 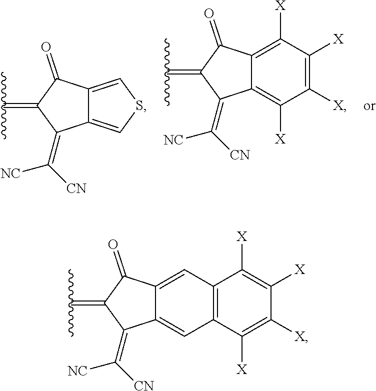
X in R₃ being at least one selected from the group consisting of H, F, and Cl.

7. The nanoimaging agent of claim 6, wherein the organic encapsulation matrix comprises at least one selected from the group consisting of methoxypolyethylene glycol amine, 1,2-distearoyl-sn-glycero-3-phosphoethanolamine-poly(ethylene glycol), 1,2-distearoyl-sn-glycero-3-phosphoethanolamine-N-[maleimide(polyethylene glycol)], 1,2-distearoyl-sn-glycero-3-phosphoethanolamine-N-[folate(polyethylene glycol)], 1,2-distearoyl-sn-glycero-3-phosphoethanolamine-N-[thiol(polyethylene glycol)], 1,2-distearoyl-sn-glycero-3-phosphoethanolamine-N-[carboxy(polyethylene glycol)], 1,2-distearoyl-sn-glycero-3-phosphoethanolamine-N-[azido(polyethylene glycol)], 1,2-distearoyl-sn-glycero-3-ethanolamine-N-[biotin(polyethylene glycol)], 1-palmitoyl-2-oleoyl-sn-glycero-3-phosphoethanolamine, 1-stearoyl-2-oleoyl lecithin, 1,2-dipalmitoyl phosphoethanolamine-polyethylene glycol, polystyrene-grafted-poly(ethylene glycol), methoxy PEG polylactic acid-hydroxyacetic acid copolymer, and Pluronic® F-127.

8. The nanoimaging agent of claim 6, wherein the nanoimaging agent is prepared by a method comprising the steps of
mixing the organic encapsulation matrix, the NIR-II organic fluorescent compound having the structure represented by formula I, and an organic solvent to obtain a mixed solution; and mixing the mixed solution with water, and subjecting a resulting mixture to ultrasonic assembly, to obtain an assembly liquid;
introducing the assembly liquid into a dialysis bag and performing dialysis, to obtain a purified assembly material; and
concentrating the purified assembly material, to obtain a solution of the nanoimaging agent.

9. The nanoimaging agent of claim 8, wherein a mass ratio of the organic encapsulation matrix to the NIR-II organic fluorescent compound having the structure represented by formula I is in a range of 3:1 to 8:1; and
the dialysis bag has a molecular weight cut-off of 3500, and the dialysis is performed for 48-72 h.

10. The nanoimaging agent of claim 8, wherein the ultrasonic assembly is performed under an ultrasonic power of 100-200 W for 3-10 min.

11. A method for providing vascular fluorescence imaging, comprising using the nanoimaging agent of claim 6.

12. The method of claim 11, wherein the vascular fluorescence imaging is provided under excitation of white light, the white light having a wavelength of 400-800 nm.

13. The nanoimaging agent of claim 7, wherein the nanoimaging agent is prepared by a method comprising the steps of
mixing the organic encapsulation matrix, the NIR-II organic fluorescent compound having the structure represented by formula I, and an organic solvent to obtain a mixed solution; and mixing the mixed solution with water, and subjecting a resulting mixture to ultrasonic assembly, to obtain an assembly liquid;
introducing the assembly liquid into a dialysis bag and performing dialysis, to obtain a purified assembly material; and
concentrating the purified assembly material, to obtain a solution of the nanoimaging agent.

14. The nanoimaging agent of claim 9, wherein the ultrasonic assembly is performed under an ultrasonic power of 100-200 W for 3-10 min.

15. The nanoimaging agent of claim 8, wherein the organic solvent is tetrahydrofuran.

16. The method of claim 11, wherein the vascular fluorescence imaging is in vivo imaging.

17. The method of claim 11, wherein the method comprises
injecting the nanoimaging agent into a subject through a vein, and performing fluorescence imaging under white light excitation.

18. The method of claim 17, wherein the nanoimaging agent injected into the subject during the vascular fluorescence imaging has an effective concentration of not less than 300 μmol·L⁻¹, and a volume of not less than 100 μL.
